SEQUENCE LISTING (39 sequences)

SEQ ID NO: 1 - protein, 13 aa, Rattus norvegicus
  VARIANT 7: note = X is S or T
PTTDSTXPAPTTK

SEQ ID NO: 2 - protein, 7 aa, Synthetic construct
  VARIANT 4: note = X is S or T
FFPXPGP

SEQ ID NO: 3 - protein, 8 aa, Homo sapiens
  VARIANT 5: note = X is S or T
GVGVXETP

SEQ ID NO: 4 - protein, 19 aa, Synthetic construct
  VARIANT 4: note = X is S or T
AAAXPAPWHEREXISSORT

SEQ ID NO: 5 - protein, 13 aa, Homo sapiens
  VARIANT 7: note = X is S or T
APALQPXQGAMPA

SEQ ID NO: 6 - protein, 13 aa, Rattus norvegicus
PTTDSTTPAPTTK

SEQ ID NO: 7 - protein, 13 aa, Rattus norvegicus
PTTDSTSPAPTTK

SEQ ID NO: 8 - protein, 7 aa, Synthetic construct
FFPTPGP

SEQ ID NO: 9 - protein, 7 aa, Synthetic construct
FFPSPGP

SEQ ID NO: 10 - protein, 8 aa, Homo sapiens
GVGVTETP

SEQ ID NO: 11 - protein, 7 aa, Homo sapiens
AAATPAP

SEQ ID NO: 12 - protein, 8 aa, Synthetic construct
GVGVSETP

SEQ ID NO: 13 - protein, 7 aa, Synthetic construct
AAASPAP

SEQ ID NO: 14 - protein, 13 aa, Homo sapiens
APALQPTQGAMPA

SEQ ID NO: 15 - protein, 13 aa, Homo sapiens
APALQPSQGAMPA

SEQ ID NO: 16 - protein, 9 aa, Synthetic construct
SSFFPTPGP

SEQ ID NO: 17 - protein, 14 aa, Synthetic construct
FFPTPGPFFPTPGP

SEQ ID NO: 18 - protein, 12 aa, Synthetic construct
SSGGGFFPTPGP

SEQ ID NO: 19 - protein, 19 aa, Synthetic construct
SSGGGFFPTPGPFFPTPGP

SEQ ID NO: 20 - protein, 13 aa, Homo sapiens
SSGGGGVGVTETP

SEQ ID NO: 21 - protein, 17 aa, Homo sapiens
GGGGAPALQPTQGAMPA

SEQ ID NO: 22 - protein, 12 aa, Synthetic construct
SSGGGAAATPAP

SEQ ID NO: 23 - protein, 145 aa, Lama glama
MDVQLQASGGGLVQAGGSLRLSCAASGFKITHYTMGWFRQAPGKEREFVSRITWGGDNTFYSNSVKGRFTISRDNAKNTVYLQMNSLKPEDTADYYCAAGSTSTATPLRVDYWGKGTQVTVSSPTTDSTTPAPTTKGGGHHHHHH

SEQ ID NO: 24 - protein, 165 aa, Lama glama
MEFGLSWLFLVAILKGVQCDVQLQASGGGLVQAGGSLRLSCAASGFKITHYTMGWFRQAPGKEREFVSRITWGGDNTFYSNSVKGRFTISRDNAKNTVYLQMNSLKPEDTADYYCAAGSTSTATPLRVDYWGKGTQVTVSSPTTDSTTPAPTTKGTGHHHHHHGT

SEQ ID NO: 25 - protein, 158 aa, Lama glama
MEFGLSWLFLVAILKGVQCDVQLQASGGGLVQAGGSLRLSCAASGFKITHYTMGWFRQAPGKEREFVSRITWGGDNTFYSNSVKGRFTISRDNAKNTVYLQMNSLKPEDTADYYCAAGSTSTATPLRVDYWGKGTQVTVSSFFPTPGPGTHHHHHHGT

SEQ ID NO: 26 - protein, 161 aa, Lama glama
MEFGLSWLFLVAILKGVQCDVQLQASGGGLVQAGGSLRLSCAASGFKITHYTMGWFRQAPGKEREFVSRITWGGDNTFYSNSVKGRFTISRDNAKNTVYLQMNSLKPEDTADYYCAAGSTSTATPLRVDYWGKGTQVTVSSGGGFFPTPGPGTHHHHHHGT

SEQ ID NO: 27 - protein, 168 aa, Lama glama
MEFGLSWLFLVAILKGVQCDVQLQASGGGLVQAGGSLRLSCAASGFKITHYTMGWFRQAPGKEREFVSRITWGGDNTFYSNSVKGRFTISRDNAKNTVYLQMNSLKPEDTADYYCAAGSTSTATPLRVDYWGKGTQVTVSSGGGFFPTPGPFFPTPGPGTHHHHHHGT

SEQ ID NO: 28 - protein, 163 aa, Lama glama
MEFGLSWLFLVAILKGVQCDVQLQASGGGLVQAGGSLRLSCAASGFKITHYTMGWFRQAPGKEREFVSRITWGGDNTFYSNSVKGRFTISRDNAKNTVYLQMNSLKPEDTADYYCAAGSTSTATPLRVDYWGKGTQVTVSSGGGGVGVTETPLGTHHHHHHGT

SEQ ID NO: 29 - protein, 163 aa, Lama glama
MEFGLSWLFLVAILKGVQCDVQLQASGGGLVQAGGSLRLSCAASGFKITHYTMGWFRQAPGKEREFVSRITWGGDNTFYSNSVKGRFTISRDNAKNTVYLQMNSLKPEDTADYYCAAGSTSTATPLRVDYWGKGTQVTVSSGGGAAATPAPGGGTHHHHHHGT

SEQ ID NO: 30 - protein, 162 aa, Lama glama
MEFGLSWVFLVAILKGVQCQVQLVESGGGLAQAGGSLRLSCAASGRTFSMDPMAWFRQPPGKEREFVAAGSSTGRTTYYADSVKGRFTISRDNAKNTVYLQMNSLKPEDTAVYYCAAAPYGANWYRDEYAYWGQGTQVTVSSGGGFFPTPGPGTHHHHHHGT

SEQ ID NO: 31 - protein, 163 aa, Lama glama
MEFGLSWVFLVAILKGVQCQVKLEESGGGLVQAGGSLRLSCAASGRTIDNYAMAWSRQAPGKDREFVATIDWGDGGARYANSVKGRFTISRDNAKGTMYLQMNNLEPEDTAVYSCAMARQSRVNLDVARYDYWGQGTQVTVSSGGGFFPTPGPGTHHHHHHGT

SEQ ID NO: 32 - protein, 384 aa, Lama glama
MEFGLSWVFLVAILKGVQCQVKLEESGGGLVQAGGSLRLSCAASGRTIDNYAMAWSRQAPGKDREFVATIDWGDGGARYANSVKGRFTISRDNAKGTMYLQMNNLEPEDTAVYSCAMARQSRVNLDVARYDYWGQGTQVTVSSEPKSSDKTHTCPPCPAPELLGGPSVFLFPPKPKDTLMISRTPEVTCVVVDVSHEGPEVKFNWHVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGGGGGFFPTPG

SEQ ID NO: 33 - protein, 391 aa, Lama glama
MEFGLSWVFLVAILKGVQCQVKLEESGGGLVQAGGSLRLSCAASGRTIDNYAMAWSRQAPGKDREFVATIDWGDGGARYANSVKGRFTISRDNAKGTMYLQMNNLEPEDTAVYSCAMARQSRVNLDVARYDYWGQGTQVTVSSEPKSSDKTHTCPPCPAPELLGGPSVFLFPPKPKDTLMISRTPEVTCVVVDVSHEGPEVKFNWHVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGGGGGAPALQPTQGAMPA

SEQ ID NO: 34 - DNA, 4497 bp, Synthetic construct
  source 1..4497: mol_type = other DNA
catatggctattggcgaattcggatcctctagagtcgacctgcagaagcttatcgatgataagctgtcaaacatgagcagatctgagcccgcctaatgagcgggcttttttttcagatctgcttgaagacgaaagggcctcgtgatacgcctatttttataggttaatgtcatgataataatggtttcttagcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgccatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtcctggcacgacaggtttcccgactggaaagcgggcagtgagcgcaacgcaattaatgtgagttagctcactcattaggcaccccaggctttacactttatgcttccggctcgtataatgtgtggaattgtgagcggataacaatttcacacaggaaacaggatcgatccatcgatgagcttactccccatccccctgttgacaattaatcatcggctcgtataatgtgtggaattgtgagcggataacaatttcacacaggaaacaggatcagcttactccccatccccctgttgacaattaatcatcggctcgtataatgtgtggaattgtgagcggataacaatttcacacaggaaacaggatccatcgatgcttaggaggt

SEQ ID NO: 35 - DNA, 4401 bp, Synthetic construct
  source 1..4401: mol_type = other DNA
gtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaacggatctctagcgaattccctctagagggcccgtttctgctagcaagcttgctagcggccgctcgaggccggcaaggccggatcccccgacctcgacctctggctaataaaggaaatttattttcattgcaatagtgtgttggaattttttgtgtctctcactcggaaggacatatgggagggcaaatcatttggtcgagatccctcggagatctctagctagaggatcgatccccgccccggacgaactaaacctgactacgacatctctgccccttcttcgcggggcagtgcatgtaatcccttcagttggttggtacaacttgccaactgaaccctaaacgggtagcatatgcttcccgggtagtagtatatactatccagactaaccctaattcaatagcatatgttacccaacgggaagcatatgctatcgaattagggttagtaaaagggtcctaaggaacagcgatgtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgataagctgtcaaacatgagaattaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagc

SEQ ID NO: 36 - protein, 219 aa, Mus musculus
DIVMSQSPSSLAVSAGERVTMSCKSSQSLLNSRTRKNYLAWYQQKPGQSPKLLIYWASTRESGVPDRFTGSGSGTDFTLTISSVQAEDLAVYYCIQSYHLRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 241 aa, Mus musculus
QVQLQQPGAELGKPGTSVKLSCKASGYTFTSYWIHWVKQRPGQGLEWVGNINPNSGSINYNEKFKNKATLTVDKSSSTAYMQLSTLTSEDFAVYYCTRDTSGQYYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGGAPALQPTQGAMPA

SEQ ID NO: 38 - protein, 29 aa, Rattus norvegicus
GWTLNSAGYLLGPHAIDNHRSFSDKHGLT

SEQ ID NO: 39 - protein, 32 aa, Rattus norvegicus
GWTLNSAGYLLGPHAIDNHRSFSDKHGLTCYA

(19) United States
(12) Patent Application Publication
(10) Pub. No.: US 2025/0101105 A1
(43) Pub. Date: Mar. 27, 2025

(54) Antibodies with Engineered O-Glycosylation Sequons for Site-Specific Conjugation

(71) Applicant: National Research Council of Canada, Ottawa (CA)

(72) Inventors: Michel Gilbert, Ottawa (CA); Yves Durocher, Montreal (CA); Warren Wakarchuk, Edmonton (CA)

(73) Assignee: National Research Council of Canada, Ottawa, ON (CA)

(21) Appl. No.: 18/848,833

(22) PCT Filed: Mar. 21, 2023
(86) PCT No.: PCT/CA2023/050365

§ 371 Date: Sep. 19, 2024

Related U.S. Application Data
(60) Provisional application No. 63/321,877, filed on Mar. 21, 2022.

(51) Int. Cl. C07K 16/28

(52) U.S. Cl. CPC C07K 16/2809; C07K 2317/41; C07K 2317/55; C07K 2317/569; C07K 2319/91

(57) ABSTRACT

The present document describes antibodies or antigen-binding fragments that bind specifically to a target antigen, wherein the antibodies or antigen-binding fragments comprise complementarity determining regions (CDR1, CDR2 and CDR3) and an added O-glycosylation sequon glycosylated with an O-glycan having the general formula (I): wherein R¹ is an initial N-acetylgalactosamine (GalNAc); n=1, or 2; R² are each independently absent, galactose (Gal), GalNAc, N-Acetylglucosamine (GlcNAc), or a sialic acid; R³ are each independently absent, Gal or a sialic acid; and R4 are each independently absent or a sialic acid. Also described are compounds comprising the antibodies or antigen-binding fragments comprising a functional moiety operably linked to said O-glycan, compositions comprising the same, and methods of preparing such compounds.

### CROSS-REFERENCE TO RELATED APPLICATIONS

This application claims priority of U.S. Provisional Patent Application No. 63/321,877 filed on Mar. 21, 2022, the specification of which is hereby incorporated by reference in its entirety.

### BACKGROUND

### (a) Field

The subject matter disclosed generally relates to antibodies or antigen-binding fragments that bind specifically to target antigens. More specifically, the subject matter relates to antibodies or antigen-binding fragments that bind specifically to target antigens and comprise complementarity determining regions (CDR1, CDR2 and CDR3) and an added O-glycosylation sequon glycosylated with an O-glycan, as well as compounds, pharmaceutical compositions, nucleic acids and nucleic acid vectors, cells comprising the nucleic acid vectors, and methods of glycosylating antibodies or antigen-binding fragments that bind specifically to target antigens and having an added O-glycosylation sequon.

### (b) Related Prior Art

Many therapeutic agents are unable to address the central nervous system indications because they cannot cross the blood-brain barrier (BBB). Enabling delivery into the brain can be achieved by linking or fusing the therapeutic agents with single-domain antibodies (sdAbs), that are called BBB carriers because they can cross the BBB by receptor-mediated transcytosis (Abulrob et al., 2005). However, an optimal process for conjugating a payload to BBB carriers requires 1) Site-specific incorporation of the payload to avoid interference with binding to the target receptor and 2) homogeneous product to facilitate approval by regulatory agencies. Current methods for linking payloads with BBB carriers are usually not site-specific enough to avoid interfering with receptor targeting and also result in populations of payload/carrier with heterogenous ratios.

In conventional conjugation chemistries, the drug molecules are attached to antibody amino acid residues (i.e., typically lysine or cysteine) using bi-functional linkers with appropriate reactive groups. In the case where the therapeutic agents are enzymes (e.g., for enzyme replacement therapies), there is the option to design genetic constructs to produce fusion proteins.

Conventional chemistries lack specificity and can interfere with targeting if the incorporation happens in a region involved in the binding to the target. Multiple reacting residues also result in heterogeneous incorporation and difficulties in obtaining an optimal payload/antibody ratio. Another issue of heterogenous products is the difficulty of getting approval from regulatory agencies. In the case of enzyme-antibody conjugates, an alternative to chemical conjugation is the production of fusion proteins. However, fusion proteins do not always fold properly, and post-translational modifications are not always optimal (or even achievable), depending on the host used for production.

Therefore, there is a need for additional means of conjugating antibodies or antigen-binding fragments that bind specifically to target antigens to payloads in a site-specific manner.

The following application describes the isolation, characterization, and in vivo testing of antibodies or antigen-binding fragments that bind specifically to target antigens and comprise complementarity determining regions (CDR1, CDR2 and CDR3) and an added O-glycosylation sequon glycosylated with an O-glycan.

### SUMMARY

According to an embodiment, there is provided an antibody or antigen-binding fragment that binds specifically to a target antigen, wherein the antibody or antigen-binding fragment comprises complementarity determining regions (CDR1, CDR2 and CDR3) and an added O-glycosylation sequon glycosylated with an O-glycan having the general formula (I):

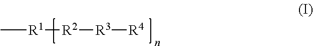

wherein
R¹ is an initial N-acetylgalactosamine (GalNAc);
n=1, or 2;
R² are each independently absent, or galactose (Gal), GalNAc, N-Acetylglucosamine (GlcNAc), or a sialic acid;
R³ are each independently absent, Gal or a sialic acid;
R⁴ are each independently absent or a sialic acid.

The initial GalNAc, and/or any one of the R¹, R², R³ and R⁴ may be further modified with one or more compound.

The compound may be one or more of a methyl group, an acetyl group, a sulfate group, or a combination thereof.

The sialic acid may be N-Acetylneuraminic acid (Neu5Ac), 9-azido-N-Acetylneuraminic acid (9N₃-Neu5Ac), N-azidoacetylneuraminic acid (Neu5NAz), or a combination thereof.

The n may be equal to 1 and R² may be Gal.

The R³ may be a sialic acid selected from the group consisting of Neu5Ac, Neu5NAz and 9N₃-Neu5Ac, and R⁴ may be absent.

The O-glycan may have the general formula (II):

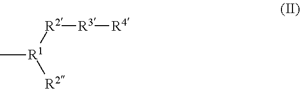

wherein
R^{2′} is Gal, or GlcNAc;
R^{3′} is Gal or a sialic acid;
R^{4′} is absent, or a sialic acid; and
R^{2″} is GlcNAc or a sialic acid.

The R^{2′} may be Gal, the R^{3′} may be a sialic acid consisting of Neu5Ac, and the R^{4′} may be absent or a sialic acid consisting of 9N₃-Neu5Ac; and the R^{2″} may be a sialic acid consisting of Neu5Ac.

The added O-glycosylation sequon may comprise an amino acid sequence comprising:
PTTDSTX₁PAPTTK, where X₁ is S or T (SEQ ID NO: 1);
FFPX₂PGP, where X₂ is S or T (SEQ ID NO: 2);
GVGVX₃ETP, where X₃ is S or T (SEQ ID NO: 3);
AAAX₄PAP, where X₄ is S or T (SEQ ID NO: 4); and
APALQPX₅QGAMPA, where X₅ is S or T (SEQ ID NO: 5), or
combinations thereof.

The added O-glycosylation sequon may comprise an amino acid sequence comprising:
PTTDSTTPAPTTK (SEQ ID NO: 6), PTTDSTSPAPTTK (SEQ ID NO: 7), FFPTPGP (SEQ ID NO: 8); FFPSPGP (SEQ ID NO: 9), GVGVTETP (SEQ ID NO: 10), GVGVSETP (SEQ ID NO: 11), AAATPAP (SEQ ID NO: 12), AAASPAP (SEQ ID NO: 13); APALQPTQGAMPA (SEQ ID NO: 14), and APALQPSQGAMPA (SEQ ID NO: 15).

The added O-glycosylation sequon may be at a C-terminus of the antibody or antigen-binding fragment.

The antigen-binding fragment may be a single-domain antibody (sdAb), a fragment antigen binding (Fab), a single-chain variable fragment (scFv), or a single-chain fragment antigen binding (scFab).

The antibody may be an IgA, an IgD, an IgE, an IgG, or an IgM.

The antibody or antigen-binding fragment may be humanized or partially humanized.

The added O-glycosylation sequon may be linked to a first functional moiety via a peptide linker.

The antibody or antigen-binding fragment is functionally linked to a first functional moiety via a peptide linker.

The peptide linker may comprise about 1 to about 40 amino acid residues.

The peptide linker may comprise the amino acid sequence (SS)ₙ, (GGG)ₙ, (GGGG)ₙ, (GGGS)ₙ, or (SSGGG)ₙ, wherein n≥1.

According to another embodiment, there is provided a compound comprising the antibody or antigen-binding fragment of the present invention, further comprising a second functional moiety operably linked to the O-glycan.

The second functional moiety may be operably linked to the O-glycan is a peptide, a polypeptide, a protein, an enzyme, a second antibody, an antibody fragment, a second antigen-binding fragment or a combination of any two or more thereof; wherein each of the antibody or antigen-binding fragment thereof and the linked peptide, polypeptide, protein, enzyme, second antibody, antibody fragment, second antigen-binding fragment, or the combination of any two or more thereof is functional.

According to another embodiment, there is provided a composition comprising the antibody or antigen-binding fragment of the present invention or the compound of the present invention, and a pharmaceutically acceptable diluent, carrier or excipient.

According to another embodiment, there is provided a nucleic acid molecule encoding an antibody or antigen-binding fragment thereof of the present invention.

According to another embodiment, there is provided a vector comprising the nucleic acid molecule of the present invention operably linked to one or more regulatory elements to allow expression of the antibody or antigen-binding fragment of the present invention in a host cell.

According to another embodiment, there is provided a cell comprising the vector of the present invention for expressing the antibody or antigen-binding fragment thereof of the present invention.

According to another embodiment, there is provided a method of glycosylating an antibody or antigen-binding fragment that binds specifically to a target antigen and having an added O-glycosylation sequon, wherein the antibody or antigen-binding fragment comprises complementarity determining regions (CDR1, CDR2 and CDR3), the method comprising step a):
a) reacting the antibody or antigen-binding fragment that binds specifically to a target antigen and having an added O-glycosylation sequon, with polypeptide N-acetylgalactosaminyltransferase 2 (ppGalNAcT2) and UDP-N-acetyl-α-D-galactosamine, to obtain an antibody or antigen-binding fragment that binds specifically to a target antigen having an added O-glycosylation sequon glycosylated with an O-glycan comprising an initial N-acetylgalactosamine (GalNAc).

The method may further comprise step b):
b) reacting the antibody or antigen-binding fragment that binds specifically to a target antigen having an added O-glycosylation sequon glycosylated with an O-glycan comprising an initial GalNAc, with a β-1,3-galactosyltransferase (β3GalT) enzyme and UDP-galactose, to obtain an antibody or antigen-binding fragment that binds specifically to a target antigen having an added O-glycosylation sequon glycosylated with an O-glycan comprising GalNAc-Gal.

The method may further comprise step c):
c) reacting the antibody or antigen-binding fragment that binds specifically to a target antigen having an added O-glycosylation sequon glycosylated with an O-glycan comprising GalNAc-Gal, with a β-Galactoside α-2,3-Sialyltransferase 1 enzyme (ST3Gal1) and CMP-9N₃-Neu5Ac, to obtain an antibody or antigen-binding fragment that binds specifically to a target antigen having an added O-glycosylation sequon glycosylated with an O-glycan comprising GalNAc-Gal-9N₃-Neu5Ac.

According to another embodiment, there is provided a method of glycosylating an antibody or antigen-binding fragment that binds specifically to a target antigen and having an added O-glycosylation sequon glycosylated with an O-glycan comprising a first sugar comprising GalNAc-Gal-Neu5Ac and a second sugar comprising NeuAc, wherein the antibody or antigen-binding fragment comprises complementarity determining regions (CDR1, CDR2 and CDR3), the method comprising step a):
a) reacting the antibody or antigen-binding fragment that binds specifically to a target antigen and having an added O-glycosylation sequon glycosylated with an O-glycan comprising a first sugar comprising GalNAc-Gal-Neu5Ac and a second sugar comprising NeuAc, with an α-N-Acetyl-Neuraminide α-2,8-Sialyltransferase enzyme and CMP-9N₃-Neu5Ac, to obtain an antibody or antigen-binding fragment that binds specifically to a target antigen having an added O-glycosylation sequon glycosylated with an O-glycan comprising a first sugar comprising GalNAc-Gal-Neu5Ac-9N₃-Neu5Ac and a second sugar comprising NeuAc.

According to another embodiment, there is provided a method of glycosylating an antibody or antigen-binding fragment that binds specifically to a target antigen and having an added O-glycosylation sequon glycosylated with an O-glycan comprising a first sugar comprising GalNAc-Gal-Neu5Ac and a second sugar comprising NeuAc, wherein the antibody or antigen-binding fragment comprises complementarity determining regions (CDR1, CDR2 and CDR3), the method comprising step a):
a) reacting the antibody or antigen-binding fragment that binds specifically to a target antigen and having an added O-glycosylation sequon glycosylated with an O-glycan comprising a first sugar comprising GalNAc-Gal-Neu5Ac and a second sugar comprising NeuAc, with a neuraminidase enzyme, to obtain an antibody or antigen-binding fragment that binds specifically to a target antigen having an added O-glycosylation sequon glycosylated with an O-glycan comprising GalNAc-Gal.

The method may further comprise step b):
b) reacting the antibody or antigen-binding fragment that binds specifically to a target antigen having an added O-glycosylation sequon glycosylated with an O-glycan comprising GalNAc-Gal, with a β-Galactoside α-2,3-Sialyltransferase 1 enzyme (ST3Gal1) and CMP-9N₃-Neu5Ac, to obtain an antibody or antigen-binding fragment that binds specifically to a target antigen having an added O-glycosylation sequon glycosylated with an O-glycan comprising GalNAc-Gal-9N₃-Neu5Ac.

According to another embodiment, there is provided a method of producing a compound comprising an antibody or antigen-binding fragment according to the present invention and a functional moiety operably linked to the O-glycan, the method comprising the step of conjugating the antibody or antigen-binding fragment comprising an O-glycan comprising a 9N₃-Neu5Ac moiety with a functional moiety comprising a functionalized cyclooctyne, to obtain a compound comprising an antibody or antigen-binding fragment conjugated to the functional moiety operably linked to the O-glycan.

The functionalized cyclooctyne may be cyclooctyne (COT), monofluorinated cyclooctyne (MOFO), difluorocyclooctyne (DIFO), dimethoxyazacyclooctyne (DIMAC), dibenzocyclooctyne (DIBO), dibenzoazacyclooctyne (DIBAC), biarylazacyclooctynone (BARAC), bicyclononyne (BCN), 2,3,6,7-tetramethoxy-DIBO (TMDIBO), sulfonylated DIBO (S-DIBO), carboxymethylmonobenzocyclooctyne (COMBO), pyrrolocyclooctyne (PYRROC), or combinations thereof.

The added O-glycosylation sequon may comprise an amino acid sequence comprising:

(SEQ ID NO: 1)
PTTDSTX₁PAPTTK, where X₁ is S or T;
(SEQ ID NO: 2)
FFPX₂PGP, where X₂ is S or T;
(SEQ ID NO: 3)
GVGVX₃ETP, where X₃ is S or T;
(SEQ ID NO: 4)
AAAX₄PAP, where X₄ is S or T;
and
(SEQ ID NO: 5)
APALQPX₅QGAMPA, where X₅ is S or T,
or
combinations thereof.

The added O-glycosylation sequon may comprise an amino acid sequence comprising:

(SEQ ID NO: 6)
PTTDSTTPAPTTK,
(SEQ ID NO: 7)
PTTDSTSPAPTTK,
(SEQ ID NO: 8)
FFPTPGP;
(SEQ ID NO: 9)
FFPSPGP,
(SEQ ID NO: 10)
GVGVTETP,
(SEQ ID NO: 11)
GVGVSETP,
(SEQ ID NO: 12)
AAATPAP,
(SEQ ID NO: 13)
AAASPAP;
(SEQ ID NO: 14)
APALQPTQGAMPA,
and
(SEQ ID NO: 15)
APALQPSQGAMPA.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### BRIEF DESCRIPTION OF THE DRAWINGS

Further features and advantages of the present disclosure will become apparent from the following detailed description, taken in combination with the appended drawings, in which:

FIG. 1 illustrates an overview of the glyco-engineering strategy used to enable site-specific linking of diagnostic of therapeutic agents to sdAbs, according to an embodiment of the present invention.

FIG. 2 illustrates the strategy for in vitro glycosylation of sdAbs with an O-sequon produced in E. coli and introduction of an azido group for conjugation, according to an embodiment of the present invention.

FIG. 3 illustrates an example of conjugation of a fluorescent label to a V_{H}H that was produced in E. coli with the EA2 mucin peptide at the C-terminus and glycosylated in vitro, according to an embodiment of the present invention. Shown is the conjugation of FC5-EA2-TAg-9N₃-NeuAc with DIBO-AF488 after in vitro O-glycosylation. Lane 1: Precision Plus Protein standards. Lane 2: FC5-EA2 without glycan; Lane 3, conjugation reaction after 2 h; Lane 4, conjugation reaction after overnight.

FIG. 4A illustrates FC5-based sdAbs with two possible O-sequons in mammalian cell lines according to an embodiment of the present invention.

FIG. 4B illustrates the strategy for the production of sdAbs with an O-sequon in mammalian cell lines and the glycan re-modeling to introduce an azido group (9N₃-NeuAc) for conjugation, according to an embodiment of the present invention. The illustrated Option 1 functions with a wider range of constructs and has more tolerance for variation of the O-glycan sequon. The illustrated Option 2 is more straightforward when the ST8NeuAc (CST-81) is active on a specific O-glycan sequon.

FIG. 5 illustrates glycan remodeling of two V_{H}H's (FC5-OG2H6 and A20.1-OG2H6) and their conjugation with PEG10K, according to an embodiment of the present invention. The figure shows a SDS-PAGE gel (performed under reducing conditions) stained with Coomassie Blue. Lanes 1, 6 and 11: molecular weight markers, lane 2: FC5-OG2H6 produced in CHO cells, lane 3: FC5-OG2H6 after removal of sialic acid using the MNV-01 neuraminidase, lane 4: FC5-OG2H6 after addition of 9N₃-NeuAc using the ST3Gal1 sialyltransferase, lane 5: FC5-OG2H6 with 9N₃-NeuAc conjugated with DBCO-PEG10K. lane 7: A20.1-OG2H6 produced in CHO cells, lane 8: A20.1-OG2H6 after removal of sialic acid using the MNV-01 neuraminidase, lane 9: A20.1-OG2H6 after addition of 9N₃-NeuAc using the ST3Gal1 sialyltransferase, lane 10: A20.1-OG2H6 with 9N₃-NeuAc conjugated with DBCO-PEG10K.

FIG. 6 illustrates the conjugation two V_{H}H's (FC5-OG2H6 and A20.1-OG2H6) with PEG10K and with MB488, according to an embodiment of the present invention. The figures show SDS-PAGE gel (performed under reducing conditions) stained with Coomassie Blue (left panel) and after visualization under long-wave UV (right panel). Lane 1: FC5-OG2H6 after glycan remodeling with 9N₃-NeuAc, Lane 2: FC5-OG2H6 with 9N₃-NeuAc conjugated with DBCO-PEG10K, Lane 3: FC5-OG2H6 with 9N₃-NeuAc conjugated with DBCO-MB488, Lane 4: molecular weight markers, Lane 5: A20.1-OG2H6 after glycan remodeling with 9N₃-NeuAc, Lane 6: A20.1-OG2H6 with 9N₃-NeuAc conjugated with DBCO-PEG10K, Lane 7: A20.1-OG2H6 with 9N₃-NeuAc conjugated with DBCO-MB488.

FIG. 7 illustrates glycan remodeling of an anti-CD3 Fab (with the OG6 sequon) and its conjugation with PEG5000, according to an embodiment of the present invention. Illustrated is the conjugation of an anti-CD3 Fab (with the OG6 sequon) with DBCO-PEG5000. The figure shows an SDS-PAGE gel (performed under reducing conditions) stained with Coomassie Blue. Lane 1: molecular weight markers, lane 2: anti-CD3 produced in CHO cells, lane 3: anti-CD3 Fab after removal of sialic acid using the MNV-02 neuraminidase, lane 4: anti-CD3 Fab after addition of 9N₃-NeuAc using the ST3Gal1 sialyltransferase, lane 5: anti-CD3 Fab with 9N₃-NeuAc conjugated with DBCO-PEG5000.

FIG. 8A shows the strategy and linker used to conjugate glycan remodeled FC5-OG2H6 with amine-reactive horse radish peroxidase (HRP).

FIG. 8B shows an SDS-PAGE analysis of fractions after separation on Superose 12 of the conjugation reaction [that was first purified by Immobilized-Metal—Affinity Chromatography (IMAC)]. Lane 1: molecular weight (MW) markers, Lanes 2, 3 and 4 contained conjugates with ratios higher than 1:1, Lanes 5 and 6 contained conjugate with 1:1 ratio and were pooled together.

FIG. 8C shows an in vitro multiple reaction monitoring (MRM) assessment of BBB transcytosis of the FC5-HRP conjugate compared with HRP alone and a positive control for crossing the BBB.

FIG. 9A illustrates the conjugation of V_{H}H-Fc fusion (IR5hFc) with an enzyme (IDS) and the demonstration that the conjugate can cross the blood brain barrier by transcytosis. Provided are details about the linkers used to conjugate the IR5hFc fusion with IDS.

FIG. 9B shows an SDS-PAGE gel showing the migration of the individual molecules and of the conjugate forms. Lane 1: IR5hFc fusion with the TCO linker, Lane 2: conjugates forms after clicking of the IR5hFc fusion (TCO linker) and the IDS (tetrazine linker), Lane 3: IDS with the tetrazine linker, Lane 4: MW markers.

FIG. 9C shows that in vitro transcytosis of IR5hFc(2)-P16-IDS(1) was evaluated in rat SV-ARBEC cells. Transport was significantly increased relative to A20.1 (negative control). The IDS bearing conjugate exhibited reduced transport relative to the IR5-hFc fusion that only carried the linker.

FIG. 10 illustrates the conjugation of FC5-OG2H6 with a peptide (rat galanin). Lane 1: FC5-OG2H6 after glycan remodeling with 9N₃-NeuAc, Lane 2: FC5-OG2H6-galanin conjugate (0.2 μg), Lane 3: FC5-OG2H6-galanin conjugate (0.5 μg), Lane 4: FC5-OG2H6-galanin conjugate (1 μg), Lane 5: MW markers.

### DETAILED DESCRIPTION

The present invention is directed to a technology for the site-specific conjugation of a payload or functional moiety, such as a drug, a protein or an enzyme, for example, to an antibody or antigen-binding fragment that binds specifically to a target antigen.

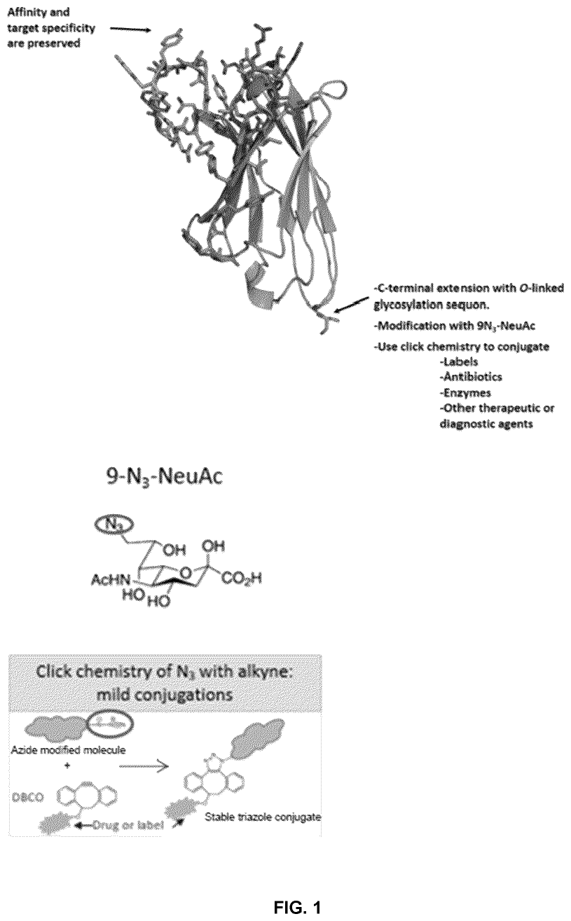

Now referring to FIG. 1, illustrates the general strategy of the present invention. FIG. 1 illustrates an embodiment where the C-terminus of sdAbs is located away from complementarity-determining regions (CDRs) which allows the introduction of an O-glycosylation site (a sequon) without interfering with target antigen recognition and affinity.

In embodiment, there is disclosed an antibody or antigen-binding fragment that binds specifically to a target antigen, wherein the antibody or antigen-binding fragment comprises complementarity determining regions (CDR1, CDR2 and CDR3) and an added O-glycosylation sequon glycosylated with an O-glycan. In embodiment, the added O-glycosylation sequon is glycosylated with an O-glycan of general formula (I):

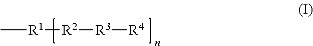

wherein
R¹ is an initial N-acetylgalactosamine (GalNAc);
n=1, or 2;
R² are each independently absent, or galactose (Gal), GalNAc, N-Acetylglucosamine (GlcNAc), or a sialic acid;
R³ are each independently absent, Gal or a sialic acid;
R⁴ are each independently absent or a sialic acid.

According to an embodiment of the present invention, the antibody or antigen-binding fragment comprises an added O-glycosylation sequon, to be glycosylated with an O-glycan. As used herein, the term “sequon” refers to the sequence of amino acids required for glycosylation, in this instant case, O-glycosylation. Proteins, antibodies and antigen-binding fragment included may comprise naturally occurring sequons. Therefore, as used herein, the sequon comprised in the invention is an added O-glycosylation sequon, found in addition to any other sequon that may be present in the antibody or antigen-binding fragment of the invention.

In embodiments, the added O-glycosylation sequon may comprise an amino acid sequence comprising PTTDSTX₁PAPTTK, where X₁ is S or T (SEQ ID NO: 1); FFPX₂PGP, where X₂ is S or T (SEQ ID NO: 2); GVGVX₃ETP, where X₃ is S or T (SEQ ID NO: 3); AAAX₄PAP, where X₄ is S or T (SEQ ID NO: 4); and APALQPX₅QGAMPA, where X₅ is S or T (SEQ ID NO: 5), or combinations thereof.

In embodiments, the added O-glycosylation sequon may comprise an amino acid sequence comprising PTTDSTTPAPTTK (SEQ ID NO: 6), PTTDSTSPAPTTK (SEQ ID NO: 7), FFPTPGP (SEQ ID NO: 8); FFPSPGP (SEQ ID NO: 9), GVGVTETP (SEQ ID NO: 10), GVGVSETP (SEQ ID NO: 11), AAATPAP (SEQ ID NO: 12), AAASPAP (SEQ ID NO: 13); APALQPTQGAMPA (SEQ ID NO: 14), and APALQPSQGAMPA (SEQ ID NO: 15).

The different O-sequons that were tried in this study are listed in Table 1.

TABLE 1
Sequons

| SEQ ID NO: | Sequence | Description |
|---|---|---|
| SEQ ID NO: 6 | PTTDSTTPAPTTK | Sequon EA2: aa 153-165 of rat Muc10-based |
| | | on sequon naturally present in mucin |
| SEQ ID NO: 8 | FFPTPGP | a synthetic sequon based on the work |
| | | published by Gerken et al., 2006 to optimize in |
| | | vitro O-glycosylation |
| SEQ ID NO: 10 | GVGVTETP | Sequon from interferon alpha 2 |
| SEQ ID NO: 12 | AAATPAP | shortest sequence motif for high level mucin- |
| | | type O-glycosylation based on Yoshida et al., |
| | | 1997 |
| SEQ ID NO: 14 | APALQPTQGAMPA | O-linked sequon present in G-CSF |
| SEQ ID NO: 16 | SSFFPTPGP | Sequon OG1: SS linker and sequon of SEQ ID |
| | | NO: 8 |
| SEQ ID NO: 17 | FFPTPGPFFPTPGP | Two sequons of SEQ ID NO: 8 in tandem |
| SEQ ID NO: 18 | SSGGGFFPTPGP | Sequon OG2: SSGGG linker and sequon of |
| | | SEQ ID NO: 8 |
| SEQ ID NO: 19 | SSGGGFFPTPGPFFPTPGP | Sequon OG3: SSGGG linker and two sequons |
| | | of SEQ ID NO: 8 in tandem |
| SEQ ID NO: 20 | SSGGGGVGVTETP | Sequon OG4: SSGGG linker and sequon of |
| | | SEQ ID NO: 10 (sequon from interferon alpha 2) |
| SEQ ID NO: 21 | GGGGAPALQPTQGAMPA | Sequon OG6: GGGG linker and sequon of SEQ |
| | | ID NO: 14 (O-linked sequon present in G-CSF) |
| SEQ ID NO: 22 | SSGGGAAATPAP | Sequon OG5: SSGGG linker and sequon of |
| | | SEQ ID NO: 12 (shortest sequence motif for |
| | | high level mucin-type O-glycosylation based on |
| | | Yoshida et al., 1997) |

In embodiment, the added O-glycosylation sequon may be at a C-terminus of the antibody or antigen-binding fragment. According to another embodiment, the O-glycosylation sequon may be at the N-terminus of the antibody or antigen-binding fragment, within the sequence of the antibody or antigen-binding fragment, at the C-terminus of the antibody or antigen-binding fragment, or combinations thereof.

In embodiment, as detailed above, the added O-glycosylation sequon is glycosylated with an O-glycan of general formula (I):

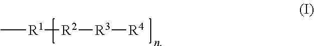

In embodiments, the R¹ is an initial N-acetylgalactosamine (GalNAc), where n=1, or 2. R² may each independently be absent, or galactose (Gal), GalNAc, N-Acetylglucosamine (GlcNAc), or a sialic acid. R³ may each independently be absent, Gal or a sialic acid. R⁴ may each independently be absent or a sialic acid. According to another embodiment, n may be equal to 1 and R² may be Gal. According to yet another embodiment, the R³ may be a sialic acid selected from the group consisting of Neu5Ac and 9N₃-Neu5Ac, and R⁴ may be absent.

In embodiment, the initial GalNAc, and/or any one of the R¹, R², R³ and R⁴ may be further modified with one or more compound. For example, the compound may be one or more of a methyl group, an acetyl group, a sulfate group, or a combination thereof.

According to an embodiment, the O-glycan may have the general formula (II):

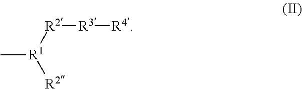

In the general formula (II), the R^{2′} may be Gal, or GlcNAc. The R^{3′} may be Gal or a sialic acid. The R^{4′} may be absent, or a sialic acid. The R^{2″} may be GlcNAc or a sialic acid.

According to another embodiment, the R^{2′} may be Gal, the R^{3′} may be a sialic acid consisting of Neu5Ac, and the R^{4′} may be absent or a sialic acid consisting of 9N₃-Neu5Ac; and the R^{2″} may be a sialic acid consisting of Neu5Ac.

In embodiments, sialic acids are a class of alpha-keto acid sugars with a nine-carbon backbone found widely distributed in animal tissues and related forms are found to a lesser extent in other organisms like in some micro-algae, bacteria and archaea. Sialic acids are commonly part of glycoproteins, glycolipids or gangliosides, where they decorate the end of sugar chains at the surface of cells or soluble proteins. According to embodiments of the present invention, the sialic acid may be N-Acetylneuraminic acid (Neu5Ac), 9-azido-N-Acetylneuraminic acid (9N₃-Neu5Ac), N-azidoacetylneuraminic acid (Neu5NAz), or a combination thereof.

According to an embodiment of the present invention, the antibody or antigen-binding fragment may be a single-domain antibody (sdAb), a fragment antigen binding (Fab), a single-chain variable fragment (scFv), or a single-chain fragment antigen binding (scFab). The antibody or antigen-binding fragment may be an IgA, an IgD, an IgE, an IgG, or an IgM. In embodiments, the antibody or an antigen-binding fragment that specifically binds to a target antigen comprises four framework regions (FR1 to FR4) and three complementarity determining regions (CDR1, CDR2 and CDR3).

As used herein, the expression “substantially identical sequence” is intended to mean an amino acid sequence which may comprise one or more conservative amino acid mutations. It is known in the art that the introduction of one or more conservative amino acid mutations to a reference sequence may yield a mutant peptide with no substantial change in physiological, chemical, physico-chemical or functional properties compared to the reference sequence. In such a case, the reference and mutant sequences would be considered “substantially identical” polypeptides. A conservative amino acid substitution is defined herein as the substitution of an amino acid residue for another amino acid residue with similar chemical properties (e.g., size, charge, or polarity). According to one embodiment, one or more conservative amino acid mutations may be made to the one or more framework regions of the sdAb while maintaining both the CDR sequences and the overall structure of the CDR of the antibody or antigen-binding fragment; thus the specificity and binding of the antibody or antigen-binding fragment are maintained. According to another embodiment, one or more conservative amino acid mutations may be made to the one or more framework regions of the sdAb and to a CDR sequence while maintaining the antigen-binding function of the overall structure of the CDR of the antibody or antigen-binding fragment; thus the specificity and binding of the antibody or antigen-binding fragment are maintained.

In a non-limiting example, a conservative mutation may be a conservative amino acid substitution. Such a conservative amino acid substitution may substitute a basic, neutral, hydrophobic, or acidic amino acid for another amino acid of the same group. By the term “basic amino acid” it is meant a hydrophilic amino acid having a side chain pK value of greater than 7, which is typically positively charged at physiological pH. Basic amino acids include histidine (His or H), arginine (Arg or R), and lysine (Lys or K). By the term “neutral amino acid” (also “polar amino acid”), it is meant a hydrophilic amino acid having a side chain that is uncharged at physiological pH, but which has at least one bond in which the pair of electrons shared in common by two atoms is held more closely by one of the atoms. Polar amino acids include serine (Ser or S), threonine (Thr or T), cysteine (Cys or C), tyrosine (Tyr or Y), asparagine (Asn or N), and glutamine (Gln or Q). By the term “hydrophobic amino acid” (also “non-polar amino acid”) it is meant an amino acid exhibiting a hydrophobicity of greater than zero according to the normalized consensus hydrophobicity scale of Eisenberg (1984). Hydrophobic amino acids include proline (Pro or P), isoleucine (Ile or I), phenylalanine (Phe or F), valine (Val or V), leucine (Leu or L), tryptophan (Trp or W), methionine (Met or M), alanine (Ala or A), and glycine (Gly or G). “Acidic amino acid” refers to a hydrophilic amino acid having a side chain pK value of less than 7, which is typically negatively charged at physiological pH. Acidic amino acids include glutamate (Glu or E) and aspartate (Asp or D).

Sequence identity is used to evaluate the similarity of two sequences. It is determined by calculating the percentage of residues that are the same when the two sequences are aligned for maximum correspondence between residue positions. Any known method may be used to calculate sequence identity; for example, computer software is available to calculate sequence identity. Without wishing to be limiting, sequence identity can be calculated by software such as NCBI BLAST2 service maintained by the Swiss Institute of Bioinformatics (and as found at ca.expasy.org/tools/blast/), BLAST-P, Blast-N, or FASTA-N, or any other appropriate software that is known in the art.

The substantially identical sequences of the present invention may be at least 90% identical; in another example, the substantially identical sequences may be at least 90%, at least 91%, at least 92%, at least 93%, at least 94%, at least 95%, at least 96%, at least 97%, at least 98%, or at least 99% identical, or any percentage there between, at the amino acid level to sequences described herein. Importantly, a substantially identical sequence retains the activity and specificity of the reference sequence. In a non-limiting embodiment, the difference in sequence identity may be due to one or more conservative amino acid mutations. In a non-limiting example, the present invention may be directed to an antibody or antigen-binding fragment comprising a sequence at least at least 95%, at least 98%, or at least 99% identical to that of one or more of the antibodies or antigen-binding fragments described herein.

The antibody or an antigen-binding fragment of the present invention may be used, for example, to bind specifically to a target antigen. As used herein, the expression “bind specifically to a target antigen” is intended to mean that the antibody or antigen-binding fragment of the present invention is enabled to bind specifically to a given antigen. In the context of the present invention, the target antigen may be any antigen of interest for example for therapeutic purpose, diagnostic purpose, or any other purpose that may be of interest, and for which conjugation of the antibody or antigen-binding fragment to a given payload is desired.

The term “antibody”, also referred to in the art as “immunoglobulin” (Ig), as used herein refers to an antigen-binding protein constructed from paired heavy and light polypeptide chains; various Ig isotypes exist, including IgA, IgD, IgE, IgG, and IgM. When an antibody is correctly folded, each chain folds into a number of distinct globular domains joined by more linear polypeptide sequences. For example, the immunoglobulin light chain folds into a variable (V_{L}) and a constant (C_{L}) domain, while the heavy chain folds into a variable (V_{H}) and three constant (C_{H}1, C_{H}2, C_{H}3) domains. Interaction of the heavy and light chain variable domains (V_{H} and V_{L}) results in the formation of an antigen binding region (Fv). Each domain has a well-established structure familiar to those of skill in the art.

The light and heavy chain variable regions are responsible for binding a target antigen and can therefore show significant sequence diversity between antibodies. The constant regions show less sequence diversity, and are responsible for binding a number of natural proteins to elicit important biochemical events. The variable region of an antibody contains the antigen-binding determinants of the molecule, and thus determines the specificity of an antibody for its target antigen. The majority of sequence variability occurs in six hypervariable regions, three each per variable heavy (V_{H}) and light (V_{L}) chain; the hypervariable regions combine to form the antigen-binding site, and contribute to binding and recognition of an antigenic determinant. The specificity and affinity of an antibody for its antigen is determined by the structure of the hypervariable regions, as well as their size, shape, and chemistry of the surface they present to the antigen. Various schemes exist for identification of the regions of hypervariability, the two most common being those of Kabat and of Chothia and Lesk. Kabat and Wu (1991) define the “complementarity-determining regions” (CDRs) based on sequence variability at the antigen-binding regions of the V_{H} and V_{L} domains. Chothia and Lesk (1987) define the “hypervariable loops” (H or L) based on the location of the structural loop regions in the V_{H} and V_{L} domains. These individual schemes define CDR and hypervariable loop regions that are adjacent or overlapping. Those of skill in the antibody art often utilize the terms “CDR” and “hypervariable loop” interchangeably, and they may be so used herein. The CDRs/loops are identified herein according to the IMGT nomenclature scheme (i.e., CDR1, 2 and 3, for each variable region).

An “antibody fragment” or “antigen-binding fragment” as referred to herein may include any suitable antigen-binding antibody fragment known in the art. The antibody fragment may be a naturally-occurring antibody fragment, or it may be a non-naturally occurring antibody fragment obtained, for example, by manipulation of a naturally-occurring antibody or by recombinant methods. For example, an antibody fragment may include, but is not limited to, a Fv, a single-chain Fv (scFv; a molecule consisting of V_{L} and V_{H} connected with a peptide linker), a Fab, a F(ab′)₂, single-domain antibody (sdAb; a fragment composed of a single V_{L} or V_{H} or a V_{H}H), or a multivalent presentation of any of these. Antibody fragments such as those just described may require one or more linker sequences, disulfide bonds, or other type of covalent bond to link different portions of the fragments; those of skill in the art will be familiar with the requirements of the different types of fragments and various approaches for their construction.

In a non-limiting example, the antigen-binding fragment of the present invention may be a sdAb derived from a naturally-occurring source. Heavy chain antibodies of camelid origin (Hamers-Casterman et al, 1993) lack light chains and thus their antigen binding sites consist of one domain, termed V_{H}H. SdAbs have also been observed in shark and are termed V_{NAR} (Nuttall et al, 2003). Other sdAbs may be engineered based on human Ig heavy and light chain sequences (Jespers et al, 2004; To et al, 2005). As used herein, the term “sdAb” includes an sdAb directly isolated from a V_{H}, V_{H}H, V_{L}, or V_{NAR} reservoir of any origin through phage display or other technology, an sdAb derived from the aforementioned sdAb, a recombinantly produced sdAb, as well as an sdAb generated through further modification of such sdAb by humanization, affinity maturation, stabilization, solubilization, camelization, or other methods of antibody engineering. Also encompassed by the present invention are homologues, derivatives, or fragments that retain the antigen-binding function and specificity of the sdAb.

SdAbs possess desirable properties for antibody molecules, such as high thermostability, high detergent resistance, relatively high resistance to proteases (Dumoulin et al, 2002) and high production yield (Arbabi-Ghahroudi et al, 1997). They can also be engineered to have very high affinity by isolation from an immune library (Li et al, 2009) or by in vitro affinity maturation (Davies & Riechmann, 1996). Further modifications to increase stability, such as the introduction of one or more non-canonical disulfide bonds (Hussack et al, 2011a,b; Kim et al, 2012), may also be brought to the sdAb.

A person of skill in the art would be well-acquainted with the structure of a single-domain antibody (see, for example, 3DWT, 2P42 in Protein Data Bank). An sdAb comprises a single immunoglobulin domain that retains the immunoglobulin fold; most notably, only three CDR/hypervariable loops form the antigen-binding site. However, and as would be understood by those of skill in the art, not all CDRs may be required for binding the antigen. For example, and without wishing to be limiting, one, two, or three of the CDRs may contribute to binding and recognition of the antigen by the sdAb of the present invention. The CDRs of the sdAb or variable domain are referred to herein as CDR1, CDR2, and CDR3.

The present invention further encompasses an antibody or an antigen-binding fragment that is “humanized” using any suitable method known in the art, such as, but not limited to, CDR grafting or veneering. Humanization of an antibody or an antigen-binding fragment comprises replacing an amino acid in the antibody or antigen-binding fragment sequence with its human counterpart, as found in the human consensus sequence, without substantial loss of antigen-binding ability or specificity; this approach reduces immunogenicity of the antibody or antigen-binding fragment when introduced into a human subject. In the process of CDR grafting, one or more than one of the CDRs defined herein may be fused or grafted to a human variable region (V_{H}, or V_{L}), to other human antibody (IgA, IgD, IgE, IgG, and IgM), to a human antibody fragment framework region (Fv, scFv, Fab) or to another protein of similar size and nature onto which a CDR can be grafted (Nicaise et al, 2004). In such a case, the conformation of the one or more than one hypervariable loop is likely preserved, and the affinity and specificity of the antibody or antigen-binding fragment for its target (i.e., a target antigen) is likely minimally affected. CDR grafting is known in the art and is described in at least the following: U.S. Pat. Nos. 6,180,370, 5,693,761, 6,054,297, 5,859,205, and European Patent No. 626390. Veneering, also referred to in the art as “variable region resurfacing”, involves humanizing solvent-exposed positions of an antibody or antigen-binding fragment; thus, preserving buried non-humanized residues, which may be important for CDR conformation, while minimizing the potential for immunological reaction against solvent-exposed regions. Veneering is known in the art and is described in at least the following: U.S. Pat. Nos. 5,869,619, 5,766,886, 5,821,123, and European Patent No. 519596. Persons of skill in the art would also be amply familiar with methods of preparing such humanized antibody fragments and humanizing amino acid positions.

The antibody or antigen-binding fragment according to the present invention may comprise one or more additional sequences to aid in expression, detection or purification of the antibody or antigen-binding fragment. Any such sequence or tag known to those of skill in the art may be used. For example, and without wishing to be limiting, the antibody or antigen-binding fragment may comprise a targeting or signal sequence (such as, but not limited to, ompA or pelB), a detection/purification tag (such as, but not limited to, c-Myc, HA, His5, or His6), or a combination of any two or more thereof. In another example, the additional sequence may be a biotin recognition site such as that described in WO/1995/004069 or by Voges et al. in WO/2004/076670. As is also known to those of skill in the art, a linker sequence may be used in conjunction with the additional sequence or tag, or may serve as a detection/purification tag.

According to an embodiment, there is disclosed an antibody or antigen-binding fragment according to the present invention, linked to a functional moiety, optionally by a linker sequence. For example, and according to an embodiment, the added O-glycosylation sequon may be linked to a first functional moiety via a peptide linker, or another portion of the antibody or antigen-binding fragment may be functionally linked to a first functional moiety via a peptide linker.

In another embodiment, there is disclosed a compound comprising an antibody or antigen-binding fragment according to the present invention, linked to a functional moiety, optionally by a linker sequence. According to another embodiment, there is disclosed a compound comprising an antibody or antigen-binding fragment according to the present invention, comprising a functional moiety operably linked to the O-glycan.

In embodiments of the antibody or antigen-binding fragment according to the present invention, or of the compound, the antibody or antigen-binding fragment may be linked to the functional moiety via a linker (also known as a linker sequence). As used herein, the term “linker sequence” is intended to mean a short (typically 40 amino acids or fewer) peptide sequence that is introduced between protein domains. Linker sequences are often composed of flexible residues such as glycine and serine so that the linked protein domains are free to move relative to one another. The linker sequence can be any linker sequence known in the art that would allow for the antibody and the functional moiety of the present invention to be operably linked for the desired function. The linker may be any sequence known in the art (either a natural or synthetic linker) that allows for an operable fusion comprising an antibody or antigen-binding fragment linked to a polypeptide (e.g. the functional moiety). For example, the linker sequence may be a linker sequence L such as (SS)ₙ, (GGG)ₙ, (GGGG)ₙ, (GGGS)ₙ, or (SSGGG)ₙ, wherein n is equal to or greater than 1, or from about 1 to about 5, or from about 1 to 15; or n may be any number that would allow for the operability of the compound of the present invention. In another example, the linker may be an amino acid sequence, for example, an amino acid sequence that comprises about 1 to about 40 amino acids, or about 3 to about 40 amino acids, or about 5 to about 40 amino acids, or about 10 to about 40 amino acids, or about 15 to about 40 amino acids, or about 20 to about 40 amino acids, or about 25 to about 40 amino acids, or about 30 to about 40 amino acids, or about 35 to about 40 amino acids, or about 3 to about 35 amino acids, or about 5 to about 35 amino acids, or about 10 to about 35 amino acids, or about 15 to about 35 amino acids, or about 20 to about 35 amino acids, or about 25 to about 35 amino acids, or about 30 to about 35 amino acids, or about 3 to about 30 amino acids, or about 5 to about 30 amino acids, or about 10 to about 30 amino acids, or about 15 to about 30 amino acids, or about 20 to about 30 amino acids, or about 25 to about 30 amino acids, or about 3 to about 25 amino acids, or about 5 to about 25 amino acids, or about 10 to about 25 amino acids, or about 15 to about 25 amino acids, or about 20 to about 25 amino acids, or about 3 to about 20 amino acids, or about 5 to about 20 amino acids, or about 10 to about 20 amino acids, or about 15 to about 20 amino acids, or about 3 to about 15 amino acids, or about 5 to about 15 amino acids, or about 10 to about 15 amino acids, or about 15 to about 20 amino acids, or about 3 to about 10 amino acids, or about 5 to about 10 amino acids, or about 3 to about 5 amino acids, or up to 3, up to 5, up to 10, up to 15, up to 20, up to 25, up to 30, up to 35, or up to 40 amino acids.

As used herein, the term “functional moiety” is intended to mean a part of the compound having an activity, purpose, or task; relating to the way in which the compound is intended to work or operate. As used herein, the term “functional moiety” is related to the generic term “payload” which is referred to above as the moiety of interest to be conjugated to the antibody or antigen-binding fragment via the O-glycan linked to the added sequon.

In embodiments, the functional moiety may be linked to the antibody or antigen-binding fragment, for example, through a chemical link pursuant to a chemical reaction, through fusion of the antibody or antigen-binding fragment with the functional moiety, obtained for example using recombinant DNA technology, and/or conjugated to the antibody or antigen-binding fragment via the O-glycan linked to the added sequon.

According to an embodiment, the antibody or antigen-binding fragment of the compound may be fused to a peptide, a polypeptide (e.g. growth factor CIBP2, an antimicrobial cyclic peptide), a protein, an enzyme [such as iduronate-2-sulfatase (IDS), acid beta-glucosidase (GCase), a serine protease, a growth factor, etc.], another (or the same) antibody or a fragment operable to bind a target epitope (e.g. an anti-microbial antibody, an anti-inflammatory antibody, an intrabody, a BBB-crossing antibody, a neurodegeneration target antibody, an ion channel targeting antibody, a cancer associated antigen antibody, a checkpoint inhibitor targeting antibody, or a GPCR targeting antibody)(for any use and for example for use in imaging, diagnostic, affinity purification, etc.), or a combination of any two or more thereof, in which both the antibody or antigen-binding fragment and the rest of the compound (i.e. the functional moiety) remain functional for their intended purpose. In a preferred embodiment, the compound may be fused to a second antibody or antigen-binding fragment, operable to bind a target epitope, which may be the same as, or distinct from the epitope of the antibody or antigen-binding fragment of the present invention.

The antibody or antigen-binding fragment of the present invention may also be in a multivalent display format, also referred to herein as multivalent presentation. Multimerization may be achieved by any suitable method known in the art. For example, and without wishing to be limiting in any manner, multimerization may be achieved using self-assembly molecules such as those described in Zhang et al (2004a; 2004b) and WO2003/046560, where pentabodies are produced by expressing a fusion protein comprising the antibody or antigen-binding fragment of the present invention and the pentamerization domain of the B-subunit of an AB5 toxin family (Merritt & Hol, 1995). A multimer may also be formed using the multimerization domains described by Zhu et al. (2010); this form, referred to herein as a “combody” form, is a fusion of the antibody or fragment of the present invention with a coiled-coil peptide resulting in a multimeric molecule (Zhu et al., 2010). Other forms of multivalent display are also encompassed by the present invention. For example, and without wishing to be limiting, the antibody or antigen-binding fragment may be presented as a dimer, a trimer, or any other suitable oligomer. This may be achieved by methods known in the art (Spiess et al, 2015), for example by direct linking connection (Nielsen et al, 2000), c-jun/Fos interaction (de Kruif & Logtenberg, 1996), or “Knob into holes” interaction (Ridgway et al, 1996).

Another method known in the art for multimerization is to dimerize the antibody or antigen-binding fragment using an Fc domain, such as, but not limited to a human Fc domain. The Fc domain may be selected from various classes including, but not limited to, IgG, IgM, or various subclasses including, but not limited to IgG1, IgG2, etc. In this approach, the Fc gene is inserted into a vector along with the sdAb gene to generate a sdAb-Fc fusion protein (Bell et al, 2010; Iqbal et al, 2010); the fusion protein is recombinantly expressed, then purified. For example, and without wishing to be limiting in any manner, a multivalent display format may encompass a chimeric or humanized format of V_{H}H of the present invention linked to an Fc domain, or bi or tri-specific antibody fusions with two or three V_{H}Hs recognizing unique epitopes. Such antibodies are easy to engineer and produce, can greatly extend the serum half-life of a sdAb, and may be excellent tumor imaging reagents (Bell et al., 2010).

The Fc domain in the multimeric complex as just described may be any suitable Fc fragment known in the art. The Fc fragment may be from any suitable source; for example, the Fc fragment may be of mouse or human origin. In a specific, non-limiting example, the Fc fragment may be a mouse Fc2b fragment or a human Fc1 fragment (Bell et al, 2010; Iqbal et al, 2010). The Fc fragment may be fused to the N-terminal or C-terminal end of the V_{H}H or humanized version of the present invention.

Each subunit of the multimers described above may comprise the same or different antibodies or antigen-binding fragments of the present invention, which may have the same or different specificity. Additionally, the multimerization domains may be linked to the antibody or antigen-binding fragment using a linker, as required; such a linker should be of sufficient length and appropriate composition to provide flexible attachment of the two molecules but should not hamper the antigen-binding properties of the antibody or antigen-binding fragment. As defined above, the linker sequence can be any linker known in the art that would allow for the compound of the present invention to be prepared and be operable for the desired function.

According to another embodiment, the present invention also encompasses a composition comprising one or more than one compound as described herein. The composition may comprise a single sdAb and/or compound as described above, or the composition may comprise a mixture of sdAbs and/or compounds. Furthermore, in a composition comprising a mixture of sdAbs and/or compounds of the present invention, the sdAbs and/or compounds may have the same specificity, or they may differ in their specificities.

A composition according to the invention may also comprise a pharmaceutically acceptable diluent, excipient, or carrier. The diluent, excipient, or carrier may be any suitable diluent, excipient, or carrier known in the art that is compatible with other ingredients in the composition, that is compatible with the method of delivery of the composition, and that is not deleterious to the recipient of the composition. The composition may be in any suitable form; for example, the composition may be provided in suspension form, powder form (such as, but not limited to, lyophilised or encapsulated), capsule form or tablet form. For example, and without wishing to be limiting, when the composition is provided in suspension form, the carrier may comprise water, saline, or a suitable buffer, and optionally comprise one or more additives to improve solubility and/or stability. Reconstitution to produce a suspension may be effected in a buffer at a suitable pH to ensure the viability of the antibody or antigen-binding fragment. Dry powders may also include additives to improve stability and/or carriers to increase bulk/volume; for example, and without wishing to be limiting, the dry powder composition may comprise sucrose or trehalose. In a specific, non-limiting example, the composition may be formulated for delivery of the antibody or antigen-binding fragment to the gastrointestinal tract of the subject. Thus, the composition may comprise encapsulation, time release, or other suitable technologies for delivery of the sdAb and/or compound of the present invention. It would be within the competency of a person of skill in the art to prepare suitable compositions comprising the present sdAb and/or compound.

The invention also encompasses a nucleic acid molecule comprising a nucleotide sequence encoding an antibody, antigen-binding fragment, or compound of the present invention. The invention further comprises a vector comprising the nucleic acid molecule; a cell comprising the vector, for expressing the antibody, antigen-binding fragment, or compound of the present invention, and a cell for expressing the antibody, antigen-binding fragment, or compound of the present invention.

The invention also encompasses a method of glycosylating an antibody or antigen-binding fragment that binds specifically to a target antigen and having an added O-glycosylation sequon, wherein the antibody or antigen-binding fragment comprises complementarity determining regions (CDR1, CDR2 and CDR3), the method comprising step a) of reacting the antibody or antigen-binding fragment that binds specifically to a target antigen and having an added O-glycosylation sequon, with polypeptide N-acetylgalactosaminyltransferase 2 (ppGalNAcT2) and UDP-N-acetyl-α-D-galactosamine, to obtain an antibody or antigen-binding fragment that binds specifically to a target antigen having an added O-glycosylation sequon glycosylated with an O-glycan comprising an initial N-acetylgalactosamine (GalNAc).

The method may further comprise step b) of reacting the antibody or antigen-binding fragment that binds specifically to a target antigen having an added O-glycosylation sequon glycosylated with an O-glycan comprising an initial GalNAc, with a β-1,3-galactosyltransferase (β3GalT) enzyme and UDP-galactose, to obtain an antibody or antigen-binding fragment that binds specifically to a target antigen having an added O-glycosylation sequon glycosylated with an O-glycan comprising GalNAc-Gal.

The method may further comprise step c) of reacting the antibody or antigen-binding fragment that binds specifically to a target antigen having an added O-glycosylation sequon glycosylated with an O-glycan comprising GalNAc-Gal, with a β-Galactoside α-2,3-Sialyltransferase 1 enzyme (ST3Gal1) and CMP-9N₃-Neu5Ac, to obtain an antibody or antigen-binding fragment that binds specifically to a target antigen having an added O-glycosylation sequon glycosylated with an O-glycan comprising GalNAc-Gal-9N₃-Neu5Ac.

The invention also encompasses a method of glycosylating an antibody or antigen-binding fragment that binds specifically to a target antigen and having an added O-glycosylation sequon glycosylated with an O-glycan comprising a first sugar comprising GalNAc-Gal-Neu5Ac and a second sugar comprising NeuAc, wherein the antibody or antigen-binding fragment comprises complementarity determining regions (CDR1, CDR2 and CDR3), the method comprising step a) of reacting the antibody or antigen-binding fragment that binds specifically to a target antigen and having an added O-glycosylation sequon glycosylated with an O-glycan comprising a first sugar comprising GalNAc-Gal-Neu5Ac and a second sugar comprising NeuAc, with an α-N-Acetyl-Neuraminide α-2,8-Sialyltransferase enzyme and CMP-9N₃-Neu5Ac, to obtain an antibody or antigen-binding fragment that binds specifically to a target antigen having an added O-glycosylation sequon glycosylated with an O-glycan comprising a first sugar comprising GalNAc-Gal-Neu5Ac-9N₃-Neu5Ac and a second sugar comprising NeuAc.

The invention also encompasses a method of glycosylating an antibody or antigen-binding fragment that binds specifically to a target antigen and having an added O-glycosylation sequon glycosylated with an O-glycan comprising a first sugar comprising GalNAc-Gal-Neu5Ac and a second sugar comprising NeuAc, wherein the antibody or antigen-binding fragment comprises complementarity determining regions (CDR1, CDR2 and CDR3), the method comprising step a) of reacting the antibody or antigen-binding fragment that binds specifically to a target antigen and having an added O-glycosylation sequon glycosylated with an O-glycan comprising a first sugar comprising GalNAc-Gal-Neu5Ac and a second sugar comprising NeuAc, with a neuraminidase enzyme, to obtain an antibody or antigen-binding fragment that binds specifically to a target antigen having an added O-glycosylation sequon glycosylated with an O-glycan comprising GalNAc-Gal.

The method may further comprise step b) of reacting the antibody or antigen-binding fragment that binds specifically to a target antigen having an added O-glycosylation sequon glycosylated with an O-glycan comprising GalNAc-Gal, with a β-Galactoside α-2,3-Sialyltransferase 1 enzyme (ST3Gal1) and CMP-9N₃-Neu5Ac, to obtain an antibody or antigen-binding fragment that binds specifically to a target antigen having an added O-glycosylation sequon glycosylated with an O-glycan comprising GalNAc-Gal-9N₃-Neu5Ac.

The invention also encompasses a method of producing a compound comprising an antibody or antigen-binding fragment according to the present invention and a functional moiety operably linked to the O-glycan, the method comprising the step of conjugating the antibody or antigen-binding fragment comprising an O-glycan comprising a 9N₃-Neu5Ac moiety with a functional moiety comprising a functionalized cyclooctyne, to obtain a compound comprising an antibody or antigen-binding fragment conjugated to the functional moiety operably linked to the O-glycan.

The functionalized cyclooctyne may be cyclooctyne (COT), monofluorinated cyclooctyne (MOFO), difluorocyclooctyne (DIFO), dimethoxyazacyclooctyne (DIMAC), dibenzocyclooctyne (DIBO), dibenzoazacyclooctyne (DIBAC), biarylazacyclooctynone (BARAC), bicyclononyne (BCN), 2,3,6,7-tetramethoxy-DIBO (TMDIBO), sulfonylated DIBO (S-DIBO), carboxymethylmonobenzocyclooctyne (COMBO), pyrrolocyclooctyne (PYRROC), or combinations thereof.

The added O-glycosylation sequon may comprise an amino acid sequence comprising PTTDSTX₁PAPTTK, where X₁ is S or T (SEQ ID NO: 1); FFPX₂PGP, where X₂ is S or T (SEQ ID NO: 2); GVGVX₃ETP, where X₃ is S or T (SEQ ID NO: 3); AAAX₄PAP, where X₄ is S or T (SEQ ID NO: 4); and APALQPX₅QGAMPA, where X₅ is S or T (SEQ ID NO: 5), or combinations thereof.

The added O-glycosylation sequon may comprise an amino acid sequence comprising PTTDSTTPAPTTK (SEQ ID NO: 6), PTTDSTSPAPTTK (SEQ ID NO: 7), FFPTPGP (SEQ ID NO: 8); FFPSPGP (SEQ ID NO: 9), GVGVTETP (SEQ ID NO: 10), GVGVSETP (SEQ ID NO: 11), AAATPAP (SEQ ID NO: 12), AAASPAP (SEQ ID NO: 13); APALQPTQGAMPA (SEQ ID NO: 14), and APALQPSQGAMPA (SEQ ID NO: 15).

The present invention will be more readily understood by referring to the following examples which are given to illustrate the invention rather than to limit its scope.

### Example 1

### Production of V_{H}H's with De Novo O-Glycosylation Sites in E. coli and In Vitro Glycosylation

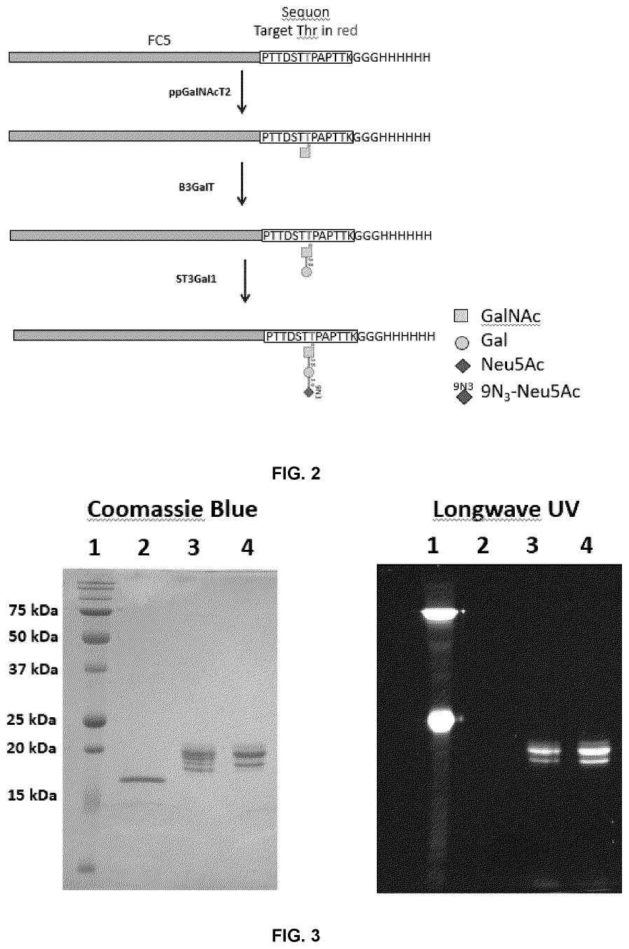

To demonstrate that sdAbs can be produced in E. coli with an O-glycosylation sequon at the C-terminus and glycosylated in vitro, the FC5 sdAb was used as the “model” sdAb. The synthetic sequons based on published work and sequences of Gerken et al. which reported an optimization of peptide sequences for in vitro O-glycosylation by ppGalNAcT2 (Gerken et al., 2006); and Yoshida et al. which determined the shortest sequence motif for high level mucin-type O-glycosylation (Yoshida et al., 1997) and the known EA2 peptide corresponds to aa 153-165 of rat Muc10 (see Table 1 for sequon sequences). The sequences were introduced at the C-terminus of FC5 to produce versions with an O-sequon at the C-terminus. The best combination of production and in vitro glycosylation was obtained using the construct FC5-15 (SEQ ID NO: 23) which has the EA2 peptide (SEQ ID NO: 1; see Table 1). The O-glycan was built enzymatically by sequentially using the ppGalNAcT2, core 1 B3GalT and ST3Gal1 (See FIG. 2). ST3Gal1 added a 9N₃-NeuAc that was used to conjugate a fluorophore (FIG. 3; right panel).

### Materials & Methods:

Production of FC5-15. The gene encoding the FC5 sdAb with the EA2 peptide and a 6-His tag was cloned in plasmid pCWori+ (SEQ ID NO:34) to give construct FC5-15 and transformed in E. coli Origami B. For production, the culture was grown at 30° C. in 2YT medium containing 150 mg/L ampicillin until A₆₀₀=0.5, induced with 0.5 mM IPTG, and then incubated for a total of 48 h, with ampicillin supplementation after 24 h (150 mg/L). The cells were resuspended in 20 mM HEPES pH 7, 0.5 M NaCl, 20 mM imidazole and Complete™ protease inhibitor. The cells were broken using an Avestin C5 Emulsiflex cell disruptor (Avestin, Ottawa, Canada) and the extract was clarified by centrifugation at 10,400×g for 20 min. FC5-15 was purified by immobilized-metal-affinity chromatography on 2×5 mL IMAC HisTrap HP columns using a 20-500 mM gradient in 20 mM HEPES pH 7, 0.5 M NaCl. FC15-15 eluted with approximately 300 mM imidazole and the fractions were pooled and concentrated by ultracentrifugation using an Ultra-4 (10 kDa MMWL) centrifugal filter units. The material was further purified by loading on 10/300 Superdex 75 HR column and eluted with 10 mM HEPES pH 7, 150 mM NaCl. The fractions were analysed by SDS-PAGE (15%) and 4.3 mg of purified FC5-15 was obtained (from a 1 L culture).

O-glycan addition to FC5-15. The α-GalNAc residue was added using the human ppGalNAcT2 expressed in E. coli(construct MPS-03). The reaction mix included 3.2 mg of FC5-15 (41 μM), 60 mU of MPS-03, 2.3 U of CPG-13 (UDP-GlcNAc/GalNAc epimerase), 20 mM Tris pH 7 and 10 mM MnCl₂ in a final volume of 5 mL. The reaction mix was incubated at 37° C. for 90 min. The addition of GalNAc was confirmed by a shift in mobility on a 15% SDS-PAGE gel. The β-1,3-galactose residue was added using the Core 1 Galactosyltransferase A from Drosophila melanogaster produced in E. coli (construct DGT-02). The reaction mix (5 mL) from the previous step was used directly for the reaction with DGT-02 (140 mU) after the addition of MES pH 6.5 (50 mM final), MnCl₂ (10 mM final) and UDP-Gal (20 mM final) in a final volume of 10 mL. The reaction mix was incubated at 37° C. for 4 h and the addition of Gal was confirmed by a shift in mobility on a 15% SDS-PAGE gel. 9-N₃-NeuAc was added using the human ST3Gal1 sialyltransferase expressed in E. coli (construct HUST-20). The reaction mix (10 mL) from the previous step was used directly after the addition of MgCl₂ (10 mM final), CMP-9N₃-NeuAc (1 mM final) and 0.5 ml of HUST-20 in a final volume of 11 mL. The reaction mix was incubated at 37° C. for 3 h and the addition of 9N₃-NeuAc was confirmed by a shift in mobility on a 15% SDS-PAGE gel. The resulting glycosylated FC5-15 sdAb was designated FC5-EA2-TAg-9N₃-NeuAc. It was purified on a 5 mL IMAC HiTrap HP column using a gradient of 10-500 mM imidazole in 10 mM HEPES pH 7, 0.5 M NaCl and we recovered 2.15 mg. The buffer was exchanged to 20 mM HEPES pH 7 by diafiltration using an Ultra-15 (10 kDa MMWL) centrifugal filter unit.

Conjugation of FC5-EA2-TAg-9N₃-NeuAc with DIBO-Alexa Fluor®488. FC5-EA2-TAg-9N₃-NeuAc (500 μg, 30.6 μmol) was conjugated with DIBO-Alexa Fluor®488 (200 nmol) in TBS buffer in a final volume of 500 μL. The reaction was incubated at room temperature for 23 h. The conjugate was purified on a PD MidiTrap G-25 column in PBS buffer pH 7.4. The degree of labeling was determined to be 1.53 using an extinction coefficient of 25,565 M⁻¹ cm⁻¹ (at 280 nm) for FC5-15 and of 71,000 M⁻¹ cm⁻¹ (at 494 nm) for the DIBO-Alexa Fluor®488.

### Discussion:

Following the workflow described in FIG. 2, an O-linked glycan was added at the C-terminus of the FC5 sdAb. This glycan was terminated with 9N₃-NeuAc which was used to click a fluorescent label (DIBO-Alexa Fluor®488). FIG. 3 shows a clear shift in mobility on SDS-PAGE after the addition of the glycan to the construct FC5-15 and clicking with DIBO-AF488. The presence of the fluorescent label is confirmed by visualization using longwave UV (FIG. 3, right panel). There is some heterogeneity in glycosylation as two major fluorescent bands are observed which correspond to the addition of either one or two glycans. The EA2 mucin sequon has multiple threonine residues and it appears that more than one of them was glycosylated in vitro.

### Example 2

### Production of V_{H}H's with De Novo O-Glycosylation Sites in Cho or Hek293 Cells and In Vitro Glycan Remodeling for Conjugation

### Introduction:

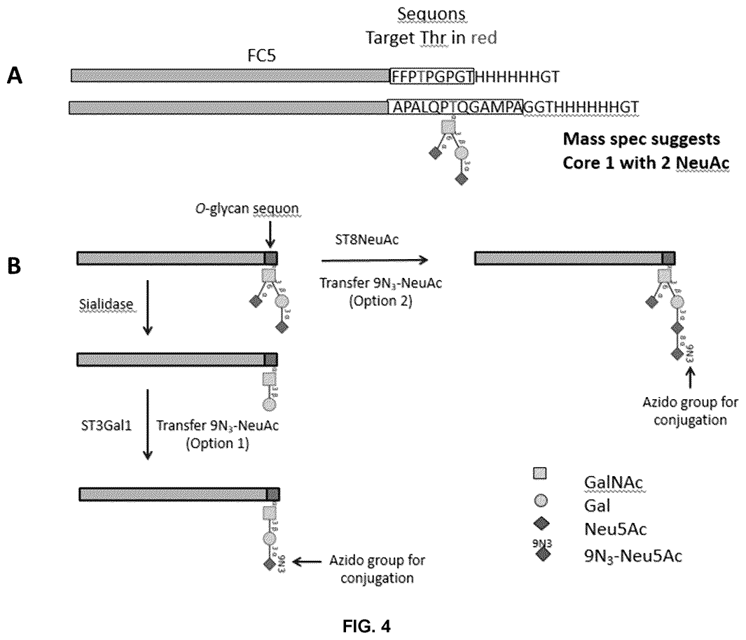

Since the in vitro glycosylation of antibodies produced in E. coli involves multiple steps that are not always quantitative, the option to produce the constructs with introduced O-glycosylation sequons in mammalian cell lines such as CHO or HEK293, was explored. The mammalian cell lines have the enzymes necessary to produce the recombinant proteins directly with the O-glycans, which can then be re-modeled to use the glycans as conjugation handles (FIGS. 4A and B).

Now referring to FIG. 4B, which illustrates the production of sdAbs with an O-sequon in mammalian cell lines and glycan re-modeling to introduce an azido group (9N₃-NeuAc) for conjugation. “Option 1” works with a wider range of constructs having more tolerance for variation of the O-glycan sequon. “Option 2” is more straightforward when the ST8NeuAc (CST-81) is active on a specific O-glycan sequon.

### Materials & Methods:

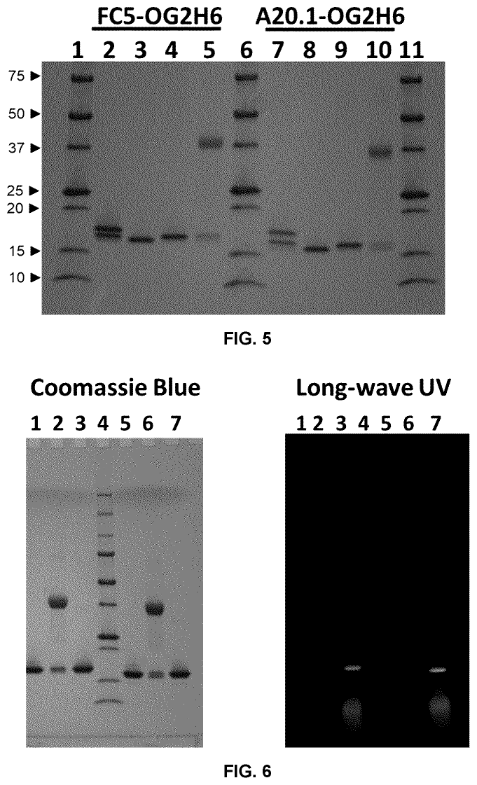

FC5-OG2H6 (SEQ ID NO: 26) and A20.1-OG2H6 (SEQ ID NO: 30) were cloned in plasmid pTT5 (SEQ ID NO:35) and produced by transient expression in CHO cells. They were purified by immobilized-metal-affinity-chromatography and size exclusion chromatography. 20 mg of each protein was digested with the MNV-01 neuraminidase for 3 h at 37° C. The neuraminidase was removed by binding FC5-OG2H6 (SEQ ID NO: 26) and A20.1-OG2H6 (SEQ ID NO: 30) to a 5 mL IMAC HisTrap HP column and eluting using a 20-500 mM gradient in 20 mM HEPES pH 7, 0.5 M NaCl. 9-N₃-NeuAc was added using the human ST3Gal1 sialyltransferase expressed in E. coli (construct HUST-20). The reaction mix included 1.3 μmol of desialylated protein, 1 mM CMP-9N₃-NeuAc, 50 mM Tris pH 7.5, 10 mM MgCl₂ and 0.3 ml of HUST-20 in a final volume of 6 mL. The reaction mix was incubated at 37° C. for 2 h and the addition of 9N₃-NeuAc was confirmed by a shift of mobility on a 15% SDS-PAGE gel as seen when comparing lane 4 with lane 3 (FC5-OGH6) and lane 9 with lane 8 (A20.1-OG2H6) in FIG. 5. The products labeled with 9-N₃-NeuAc were purified on a 5 mL IMAC HiTrap HP column using a gradient of 20-500 mM imidazole in 10 mM HEPES pH 7, 0.5 M NaCl. The buffer was exchanged to PBS by diafiltration using an Ultra-15 (10 kDa MMWL) centrifugal filter unit. FC5-OG2H6-9N₃-NeuAc and A20.1-OG2-9N₃-NeuAc (1 nmol each) were conjugated with either 4 nmol of DBCO-MB488 (final volume 10 μL containing 10% DMSO) or DBCO-PEG10K (final volume 4 μL) in PBS buffer. The reaction was incubated overnight at room temperature. The conjugation of DBCO-PEG10K was confirmed by a shift in mobility on a 15% SDS-PAGE gel while the conjugation with DBCO-MB488 was confirmed by longwave UV illumination of the gel (FIG. 6).

### Results:

Both FC5-OG2H6 (SEQ ID NO: 26) and A20.1-OG2H6 (SEQ ID NO: 30) produced in CHO cells appear as doublets (lanes 2 and 7, respectively) on 15% SDS-PAGE (FIG. 5). Mass spectrometry analysis showed that these V_{H}H's contained a mix of glycans corresponding to mono- and di-sialylated T antigen (i.e., NeuAc-α2,3-Gal-β1,3-[NeuAc-α2,6-]-GalNAcα) at the O-linked sequon (data not shown). The doublets shifted to a single band (lanes 3 and 8, respectively, in FIG. 5) with higher mobility after treating the samples with a neuraminidase, indicating the cleavage of both sialic acids. 9-N₃-NeuAc was added using the human ST3Gal1 sialyltransferase and a shift to lower mobility was observed for both V_{H}H's (lanes 4 and 9, respectively, in FIG. 5). Conjugation of DBCO-PEG10K was observed by the shift in mobility on SDS-PAGE, as observed in lanes 5 (FC5-OG2H6) and 10 (A20.1-OG2H6) of FIG. 5, and in lanes 2 (FC5-OG2H6) and 6 (A20.1-OG2H6) of FIG. 6 (left panel). Conjugation of DBCO-MB488 was confirmed by longwave UV illumination as observed in lanes 3 (FC5-OG2H6) and 7 (A20.1-OG2H6) of FIG. 6 (right panel).

### Discussion:

Mass spectrometry confirmed that both FC5-OG2H6 (SEQ ID NO: 26) and A20.1-OG2H6 (SEQ ID NO: 30) were glycosylated when they were produced in CHO cells. Glycan remodeling resulted in the removal of the natural sialic acids and replacement with 9-N₃-NeuAc. Click reactions with either DBCO-PEG10K or DBCO-MB488 confirmed that the O-glycans can be used for efficient site-specific conjugation.

### Example 3

### Conjugation of Fab's with PEG5000

### Introduction:

Since the strategy of introducing an O-linked sequon as a conjugation handle worked well for sdAbs (V_{H}H's), the extension of the application to other formats of antibodies was investigated. Firstly, the addition to an O-linked sequon at the C-terminus of the heavy chain (V_{H}-CH1) of an anti-CD3 Fab was tested, to determine if it would be produced efficiently as a glycosylated form in CHO cells.

### Materials & Methods:

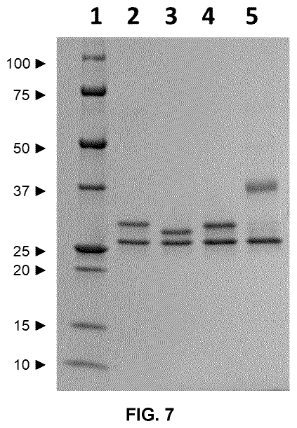

The anti-CD3 Fab (SEQ ID NO:36 and SEQ ID NO:37) with the OG6 sequon was produced by transient expression in CHO^{BRI/55E1} cells. It was purified by affinity chromatography on Capture Select CH1-XL resin (Thermo Scientific) and eluted with 50 mM sodium acetate pH 4. The anti-CD3 Fab with the OG6 sequon (20.6 mg) was digested with the MNV-02 neuraminidase (has a 6-His tag) for 3 h at 37° C. The neuraminidase was removed by binding to a 1 mL IMAC HisTrap FF column and eluting using a 20-500 mM gradient in 20 mM Hepes pH 7, 0.5 M NaCl. 9-N₃-NeuAc was added using the human ST3Gal1 sialyltransferase expressed in E. coli (construct HUST-20). The reaction mix included 31 nmol of desialylated protein, 1 mM CMP-9N₃-NeuAc, 50 mM Tris pH 7.5, 10 mM MgCl₂ and 40 μL of HUST-20 in a final volume of 1.6 mL. The reaction mix was incubated at 37° C. for 2 h and the addition of 9N₃-NeuAc was confirmed by a shift of mobility on a 15% SDS-PAGE gel, as seen by comparing lanes 3 and 4 of FIG. 7. The buffer was exchanged to 50 mM Hepes pH 7 using a 5 mL HiTrap desalting column. The product labeled with 9-N₃-NeuAc was purified by cation-exchange chromatography on a 1 mL HiTrap-SP HP column using a gradient of 0-1.0 M NaCl in 50 mM Hepes pH 7. The anti-CD3 Fab labeled with 9N₃-NeuAc (40 μmol) was conjugated with 4 nmol of DBCO-PEG5000 (final volume 4.4 μL) in 40 mM HEPES pH 7+160 mM NaCl. The reaction was incubated overnight at 4° C. The conjugation of DBCO-PEG5000 was confirmed by a shift in mobility on a 15% SDS-PAGE gel, as seen by comparing lanes 4 and 5 of FIG. 7.

### Results:

Mass spectrometry confirmed that the heavy chain of the anti-CD3 Fab with the OG6 sequon (SEQ ID NO: 21) was glycosylated with di-sialylated T antigen when it was produced in CHO cells (data not shown). SDS-PAGE showed both that the light and heavy chains had the expected molecular weights (lane 2, FIG. 7). Neuraminidase treatment resulted in a shift of the heavy chain to a form with higher mobility on a 15% SDS-PAGE gel, as seen when comparing lanes 2 and 3 of FIG. 7. The addition of 9N₃-NeuAc with ST3Gal1 caused a shift to a lower mobility form, as seen when comparing lanes 3 and 4 of FIG. 7. The conjugation of DBCO-PEG5000 was successful as confirmed by the complete shift of the heavy chain to a form with much lower mobility, as seen when comparing lanes 4 and 5 of FIG. 7.

### Discussion:

The anti-CD3 Fab with the OG6 sequon (SEQ ID NO: 21) was efficiently glycosylated when produced in CHO cells, which confirms that the strategy developed for sdAbs can be applied to Fab's. Glycan remodeling resulted in the removal of the natural sialic acids and replacement with 9-N₃-NeuAc. Click reaction with DBCO-PEG5000 confirmed that the O-glycans can be used for efficient site-specific conjugation of a Fab.

### Example 4

### Conjugation of a Glyco-Engineered V_{H}H with an Enzyme

### Introduction:

In the first 3 examples, it was shown that sdAbs (V_{H}H's) with an O-glycosylation sequon can be produced either in E. coli (for in vitro glycosylation) or in CHO cells (for in vivo glycosylation). This application was extended to Fab's produced in CHO cells and it was shown that glycan remodeling was successful to introduce a conjugation “handle” that was used to click either a fluorescent label or polyethylene glycol. In this example, it is demonstrated that the strategy can also be used to conjugate an enzyme with a V_{H}H and shown that the V_{H}H retained its functionality as a BBB carrier.

### Materials & Methods:

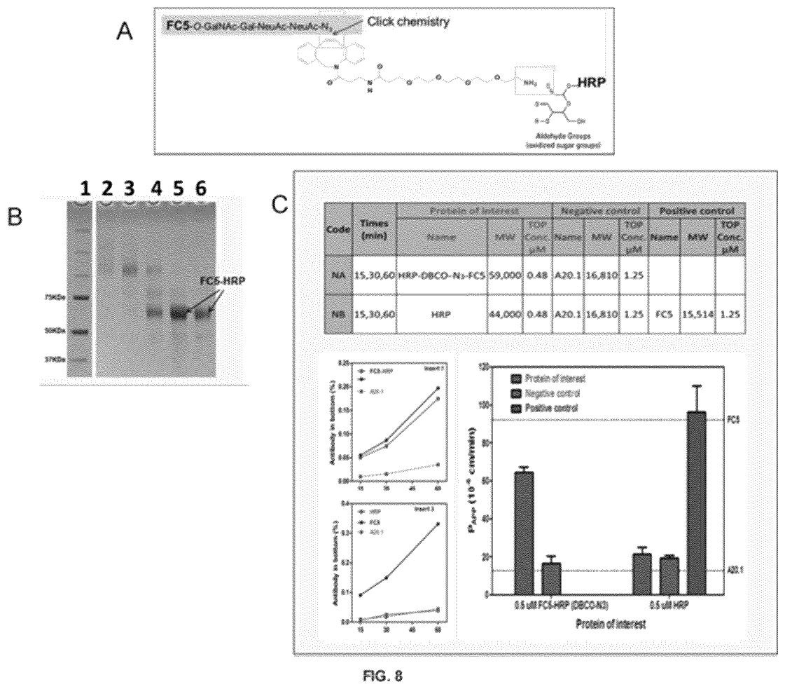

FC5-OG2H6 (SEQ ID NO: 26) was produced by transient expression in HEK293 cells. 9N₃-NeuAc was introduced directly on the O-glycan using the recombinant Campylobacter α-2,8-sialyltransferase (construct CST-81). The reaction mix included 1 mg of FC5-OG2H6 (SEQ ID NO: 26)(0.5 mg/mL), 0.36 U of CST-81, 1.5 mM CMP-9N₃-NeuAc, 50 mM Hepes pH 7.5 and 10 mM MgCl₂. The reaction was incubated overnight at 37° C., supplemented with 0.36 U of CST-81 and incubated for another 4 h at 37° C. The addition of 9N₃-NeuAc was confirmed by a shift in mobility on a 15% SDS-PAGE gel (data not shown). The resulting product was designated FC5-OG2H6-N₃. It was purified on a 1 mL IMAC HiTrap HP column using a gradient of 10-500 mM imidazole in 10 mM Hepes pH 7, 0.5 M NaCl and we recovered 0.82 mg. The buffer was exchange to PBS by diafiltration using an Amicon Ultra-4 (10 kDa MMWL) centrifugal filter unit. FC5-OG2H6-N₃ (0.5 mg) was clicked with DBCO-PEG4-amine (0.24 mM) in PBS containing 10% DMSO in a final volume of 1 mL. The conjugate of FC5-OG2H6-N₃ clicked with the DBCO-PEG4-amine was purified on a 1 mL IMAC HiTrap HP column and the buffer was exchanged to PBS using a PD MiniTrap G-25 column. The FC5-OG2H6-N₃-DBCO-PEG4-amine (0.44 mg in PBS buffer) was mixed with 1 mg of EZ-Link™ Plus Activated Peroxidase (Cat. #31487, Thermo-Fisher™) and 10 μL of 5 M sodium cyanoborohydride in a final volume of 0.33 mL. The reaction mix was incubated at 25° C. for 1 h. The reaction was then quenched with the addition of 20 μL of 3 M ethanolamine (pH 9.4) and incubated at room temperature for 15 min. The reaction mix was desalted using a PD MiniTrap G-25 column equilibrated with PBS pH 7.4. The FC5-OG2H6-HRP conjugate (See structure at FIG. 8A, and SDS-PAGE of product in lanes 5 and 6 of FIG. 8B) and was purified on a 1 mL IMAC HiTrap HP column to remove the unconjugated EZ-Link™ Plus Activated Peroxidase and by gel filtration on a Superose 12 10/300 column to remove the unconjugated FC5-OG2H6-N₃-DBCO-PEG4-amine. 106 μg of the conjugate recovered had a MW consistent with a ratio of 1:1.

### Results:

FC5OG2H6 (SEQ ID NO: 26) was produced in HEK293 cells and mass spectrometry analysis showed that it was modified with an O-linked glycan consistent with a main structure corresponding to di-sialylated T antigen. We used an α-2,8-sialyltransferase to add directly 9N₃-NeuAc that was further modified with DBCO-PEG4-amine in order to be able to conjugate directly with horseradish peroxidase using the EZ-Link™ Plus Activated Peroxidase kit. The reaction resulted in mixture of conjugation products with various ratios of conjugation partners. Gel filtration was performed to enrich a pool with 1:1 conjugates. The horseradish peroxidase-FC5 conjugate was shown to cross the BBB by transcytosis using an in vitro model (FIG. 8C).

### Discussion:

In this example, it is shown that 9N₃-NeuAc can be added directly to 0-glycans produced in HEK293 following “Option 2” (FIG. 4) that uses an α-2,8-sialyltransferase. A DBCO-PEG4-amine linker was clicked to FC5-OG2H6-9N₃-NeuAc to conjugate it to the amine-reactive EZ-Link™ Plus Activated Peroxidase. Although site-specificity was achieved for FC5-OG2H6-9N₃-NeuAc, there are multiple amine-reactive sites on the peroxidase. Gel filtration was used to enrich the 1:1 conjugate. An in vitro transcytosis model was used to show that the FC5-peroxidase conjugate could cross the membrane while the peroxidase by itself and a V_{H}H negative control could not cross the membrane (FIG. 8C).

### Example 5

### Conjugation of V_{H}H-Fc Fusions with an Enzyme: Conjugation of IR5HFC1X0OG6 with Deglycosylated IDs

### Introduction:

Since the introduction of an O-linked sequon at the C-terminus of the heavy chain of a Fab worked well as a conjugation handle (Example 3), the same strategy was employed for a V_{H}H-Fc fusion. The V_{H}H IR5 was used, rather than the ones used in the previous examples (FC5 and A20.1).

### Materials & Methods:

IR5hFc1X0OG6 (SEQ ID NO: 33) was produced by transient expression in CHO cells. The sialic acids of IR5hFc1X0OG6 (SEQ ID NO: 33) (20 mg) were removed using the MNV-01 sialidase in 50 mM MES pH 6. The reaction mix was incubated 2 h at 37° C. The de-sialylated IR5hFc1X0OG6 (SEQ ID NO: 33) was purified on a HiTrap Protein A column and desalted against PBS pH 7.4 using a PD MidiTrap G-25 column. De-sialylated IR5hFc1X0OG6 (SEQ ID NO: 33) (17.2 mg) was labelled with 9N₃-NeuAc using the ST3Gal1 sialyltransferase in a reaction mixture containing 1 mM CMP-9N₃-NeuAc, 50 mM Tris pH 7.5 and 10 mM MgCl₂. The reaction mix was incubated 2 h at 37° C. The labelled IR5hFc1X0OG6 (SEQ ID NO: 33) was purified on a HiTrap Protein A column and desalted against PBS pH 7.4 using a PD MidiTrap G-25 column. Some loss of 9N₃-NeuAc has been observed after storage at 4° C. and a second addition of 9N₃-NeuAc was performed on a 5.1 mg sample of de-sialylated IR5hFc1X0OG6 using 0.2 mM CMP-9N₃-NeuAc and ST3Gal1 in the presence of 0.4 mM of a sialidase inhibitor (DANA). The reaction was incubated 0.5 h at 37° C. and mixed with TCO-PEG12-DBCO (Click Chemistry Tools catalogue #1005-10) at a final concentration of 0.5 mM. The reaction mix was incubated 21 h at 22° C. and the buffer was then exchanged with PBS pH 7.5 containing 10 mM EDTA using an ultrafiltration unit (10 kDa MWCO). Four mg of deglycosylated IDS (batch P397 A6 #1) was reacted with bis-sulfone-PEG4-methyltetrazine (Click Chemistry Tools catalogue #1158-10) at a final concentration of 0.2 mM in PBS pH 7.5+5 mM EDTA. The reaction mix was incubated for 2 h at 22° C. and then, for 20 h at 4° C. The buffer was then exchanged with PBS pH 7.5 containing 10 mM EDTA using an ultrafiltration unit (10 kDa MWCO). The deglycosylated IDS-BSF-TZ was mixed 1:1 (71.4 nmol of each) with IR5hFc1X0OG6-N₃-DBCO-TCO and incubated 3 h at 22° C. We attempted to remove un-conjugated IR5hFc1X0OG6-N₃-DBCO-TCO by applying the sample on a HisTrap HP column, but the conjugate did not bind to the matrix, so there was essentially no purification (except for the removal of some of the unconjugated IDS-BSF-TZ). We then applied the material on a Superose 12 10/300 GL column, eluted with 20 mM HEPES pH 7.4+50 mM NaCl and pooled the fractions that were enriched in conjugates.

### Results:

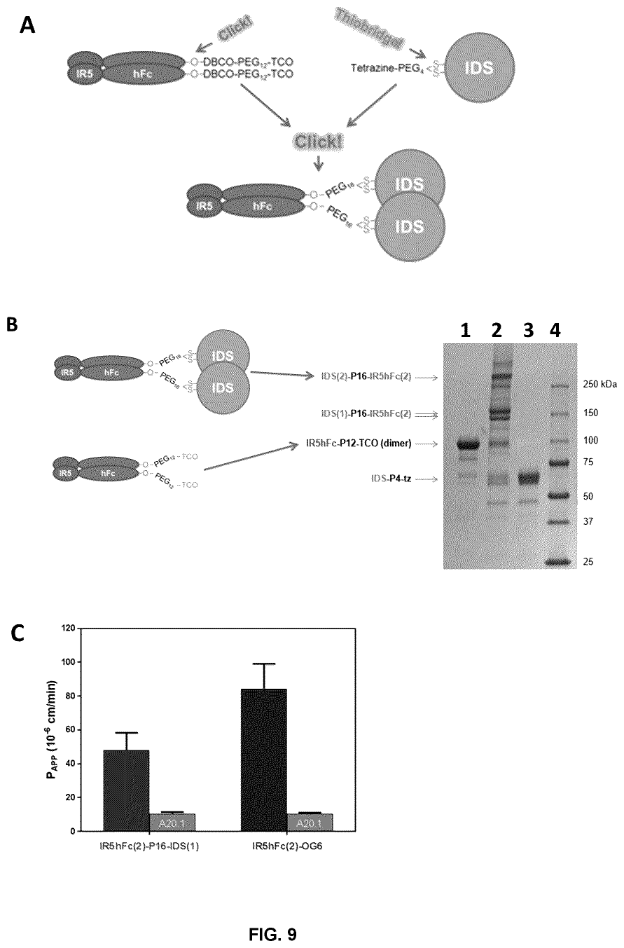

IR5hFc1X0OG6 (SEQ ID NO: 33) was produced in CHO cells and mass spectrometry analysis showed that each chain was modified with an O-linked glycan consistent with a main structure corresponding to mono- and di-sialylated T antigen (data not shown). 9N₃-NeuAc was transferred to each chain using ST3Gal1 and a TCO-PEG12-DBCO linker was clicked on the azido group. The IDS was reacted with bis-sulfone-PEG4-methyltetrazine to provide a conjugation linker on this enzyme. The conjugation of the modified IR5hFc1X0OG6 and IDS resulted in a mixture of conjugated species with either 1 or 2 IDS per IR5hFc1X0OG6 and other apparent heterogeneity observed by SDS-PAGE (FIG. 9B, lane 2). A preparation enriched in the 1:2 ratio of IDS:IR5hFc1X0OG6 was obtained by gel filtration and was used in an in vitro transcytosis assay (rat SV-ARBEC cells). The transport of the conjugated IDS was significantly increased when compared to a negative control (FIG. 9B).

### Discussion:

This example demonstrates that the introduction of O-glycosylation sequon of a V_{H}H-Fc fusion resulted in efficient in vivo O-glycosylation during production in CHO cells. 9N₃-NeuAc was added using ST3Gal1 and the azido group was modified with TCO-PEG12-DBCO linker. This linker was added in order to facilitate the conjugation with the enzyme by adding a PEG12 extension and the more reactive TCO group. The enzyme itself was reacted with bis-sulfone-PEG4-methyltetrazine in order to use the methyltetrazine group to react with the TCO group on IR5hFc1X0OG6 (FIG. 9A). Since IR5hFc1X0OG6 is a dimer, there are two TCO groups that can react and it resulted in two populations of conjugates with ratios of either 1:1 or 1:2 (FIG. 9A). More heterogeneity is suggested by SDS-PAGE analysis (FIG. 9B, lane 2). This is not surprising since it is not clear whether the bis-sulfone-PEG4-methyltetrazine reacted homogenously on the enzyme and if this will have an impact on the unfolded conformation that the conjugates will take when denatured by SDS. The BBB functionality of IR5 in the fusion (IR5hFc1X0OG6) and as part of the conjugate (IR5hFc1X0OG6-IDS) was preserved since the transport of the conjugated IDS was significantly increased when compared to a negative control (FIG. 9C).

### Example 6

### Conjugation of a Glyco-Engineered V_{H}H with a Peptide

### Introduction:

In this example, it is demonstrated that the strategy can also be used to conjugate a peptide with a glyco-engineered V_{H}H. The peptide used for this demonstration was the rat galanin with a cysteamide modification at the C-terminus for conjugation to a bifunctional linker that has a maleimide reactive group at one end and a DBCO at the other end to react with a glyco-engineered V_{H}H (FC5-OG2H6) modified with an azide.

### Materials & Methods:

A stock of 20 mM sulfo-DBCO-PEG4-maleimide (Click Chemistry Tools™, catalogue #1231-10) was prepared in dimethylformamide. Rat galanin (SEQ ID NO: 38) with a cysteamide modification at the C-terminus (SEQ ID NO: 39) was custom synthesized by Biomatik. FC5-OG2H6-9N₃-NeuAc (9 mg, 0.56 μmol) was reacted with 2.25 μmol of sulfo-DBCO-PEG4-maleimide in a final volume of 1.445 mL for 1 h at room temperature. The volume was completed to 2.5 mL by adding D-PBS (Cytiva™, catalogue #SH30028.03) and loaded on a PD-10 column (Cytiva™, catalogue #17085101) to remove the unbound linker. FC5-OG2H6-9N₃-NeuAc conjugated with the linker was then reacted with 2.25 μmol of rat galanin with a cysteamide modification at the C-terminus in a final volume of 3.36 mL for 1 h at room temperature. The material was combined with a small scale test reaction performed previously and the conjugate was purified by gel filtration on a HiLoad Superdex 75™ 16/600 pg column (Cytiva™, catalogue #28989333) equilibrated with D-PBS and eluted at 1 mL/min. Fractions containing the FC5-OG2H6-galanin conjugate were pooled and concentrated using an Amicon® Ultra-4 (10 kDa MMWL) centrifugal filter unit.

### Results:

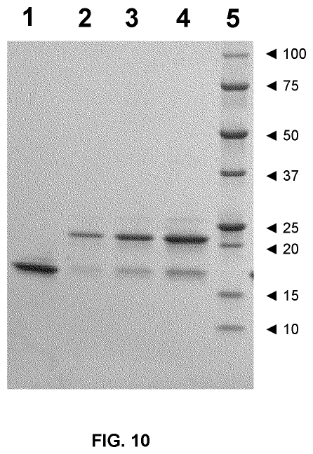

A bifunctional linker (sulfo-DBCO-PEG4-maleimide) was used to conjugate FC5-OG2H6-9N₃-NeuAc with a peptide having a cysteamide modification at the C-terminus. First the azide and DBCO were reacted to introduce the linker on the FC5-OG2H6-9N₃-NeuAc. After removing the excess linker, the cysteamide of the peptide was reacted with the maleimide of the linker attached to FC5-OG2H6-9N₃-NeuAc. Purification by gel filtration resulted in a final recovery of 8.14 mg of FC5-OG2H6-galanin conjugate. FIG. 10 shows a SDS-PAGE gel with 3 loadings of the conjugates (lanes 2-4). The shift to a lower mobility band for the conjugate (lanes 2-4), when compared with FC5-OG2H6-9N₃-NeuAc, indicated successful conjugation of the peptide. There is residual unreacted FC5-OG2H6-9N₃-NeuAc that is estimated to represent less than 20% of the material as judged by the intensity of the bands on the SDS-PAGE and by mass spectrometry (data not shown).

### Discussion:

This example provides further utility of the invention by demonstrating efficient conjugation of a glyco-engineered V_{H}H (FC5-OG2H6-9N₃-NeuAc) with a peptide (rat galanin). Galanin has analgesic and antiepileptic activity and site-specific conjugation to a BBB carrier such as FC5 provides a potential method to efficiently target this peptide to the CNS.

### Discussion of Examples

It is shown that sdAbs with O-linked sequons can be produced in E. coli and O-glycans can be synthesized in vitro using recombinant glycosyltransferases. The terminal residue (9N₃-NeuAc) included an azido group that was used to conjugate a fluorescent probe (FIG. 3). Many sdAbs (V_{H}H's) are produced in E. coli and this strategy could be used to develop versions that can be conjugated with site-specifity to molecules or nanoparticles, thus maintaining target binding and specificity. It is shown that the sdAbs including O-linked sequons can be produced in mammalian cell lines such as HEK293 and CHO. A high level of “occupancy” with di-sialylated T antigen at the O-linked sequons was observed. Glycan remodeling was performed to remove the natural sialic acids and replace them with a single 9N₃-NeuAc. It was demonstrated that the azido group can be conjugated with fluorescent labels, polyethylene glycol, enzymes and nanoparticles. The functionality of both conjugate partners was demonstrated for BBB carriers conjugated with enzymes as the conjugates were transported across a tight cell membrane in an in vitro model and the enzymes were detected across the membrane.

In addition to achieving exquisite site-specificy, the use of O-glycosylation introduces a spacer that provides additional “steric flexibility” to interact with the fusion partner. Also, the hydrophilicity helps for the solubility when the fusion partner is hydrophobic as is often the case for drugs. We have selected O-linked sequons that were used successfully with many sdAbs and antibodies and have shown utility for conjugation with a large number of conjugation partners.

While preferred embodiments have been described above and illustrated in the accompanying drawings, it will be evident to those skilled in the art that modifications may be made without departing from this disclosure. Such modifications are considered as possible variants comprised in the scope of the disclosure.

SEQUENCE TABLE

| SEQ ID NO: | Sequence | Description |
|---|---|---|
| SEQ ID NO: 1 | PTTDSTX₁PAPTTK, where X₁ is S or T | Consensus sequon EA2 |
| SEQ ID NO: 2 | FFPX₂PGP, where X₂ is S or T | Consensus sequon based on |
| | | Gerken et al. (2006) |
| SEQ ID NO: 3 | GVGVX₃ETP, where X₃ is S or T | Consensus sequon based on |
| | | sequon from interferon alpha |
| | | 2 |
| SEQ ID NO: 4 | AAAX₄PAP, where X₄ is S or T | Consensus sequon based on |
| | | Yoshida et al. (1997) |
| SEQ ID NO: 5 | APALQPX₅QGAMPA, where X₅ is S or T | Consensus sequon based on |
| | | O-linked sequon present in |
| | | G-CSF |
| SEQ ID NO: 6 | PTTDSTTPAPTTK | Sequon EA2 |
| SEQ ID NO: 7 | PTTDSTSPAPTTK | Sequon based on EA2 |
| SEQ ID NO: 8 | FFPTPGP | Synthetic sequon based on |
| | | Gerken et al. (2006) |
| SEQ ID NO: 9 | FFPSPGP | Synthetic sequon based on |
| | | Gerken et al. (2006) |
| SEQ ID NO: 10 | GVGVTETP | Sequon from interferon alpha |
| | | 2 |
| SEQ ID NO: 11 | GVGVSETP | Sequon based on sequon |
| | | from interferon alpha 2 |
| SEQ ID NO: 12 | AAATPAP | Shortest sequence motif for |
| | | high level mucin-type O- |
| | | glycosylation based on |
| | | Yoshida et al. (1997) |
| SEQ ID NO: 13 | AAASPAP | Sequon based on sequon |
| | | from Yoshida et al. (1997) |
| SEQ ID NO: 14 | APALQPTQGAMPA | O-linked sequon present in G- |
| | | CSF |
| SEQ ID NO: 15 | APALQPSQGAMPA | Sequon based on O-linked |
| | | sequon present in G-CSF |
| SEQ ID NO: 16 | SSFFPTPGP | Sequon OG1: SS linker and |
| | | sequon of SEQ ID NO: 8 |
| SEQ ID NO: 17 | FFPTPGPFFPTPGP | Two sequons of SEQ ID NO: |
| | | 8 in tandem |
| SEQ ID NO: 18 | SSGGGFFPTPGP | Sequon OG2: SSGGG linker |
| | | and sequon of SEQ ID NO: 8 |
| SEQ ID NO: 19 | SSGGGFFPTPGPFFPTPGP | Sequon OG3: SSGGG linker |
| | | and two sequons of SEQ ID |
| | | NO: 8 in tandem |
| SEQ ID NO: 20 | SSGGGGVGVTETP | Sequon OG4: SSGGG linker |
| | | and sequon from interferon |
| | | alpha 2 |
| SEQ ID NO: 21 | GGGGAPALQPTQGAMPA | Sequon OG6: GGGG linker |
| | | and O-linked sequon present |
| | | in G-CSF |
| SEQ ID NO: 22 | SSGGGAAATPAP | Sequon OG5: SSGGG linker |
| | | and shortest sequence motif |
| | | for high level mucin-type O- |
| | | glycosylation based on |
| | | Yoshida et al. (1997) |
| SEQ ID NO: 23 | MDVQLQASGGGLVQAGGSLRLSCAASGFKITHYTM | FC5-15: FC5 sdAb + EA2 |
| | GWFRQAPGKEREFVSRITWGGDNTFYSNSVKGRFT | sequon (aa 153-165 of rat |
| | ISRDNAKNTVYLQMNSLKPEDTADYYCAAGSTSTAT | Muc10) + GGG linker + poly- |
| | PLRVDYWGKGTQVTVSSPTTDSTTPAPTTKGGGHH | His-Tag |
| | HHHH | |
| SEQ ID NO: 24 | MEFGLSWLFLVAILKGVQCDVQLQASGGGLVQAGG | FC5EA2H6: Secretion signal |
| | SLRLSCAASGFKITHYTMGWFRQAPGKEREFVSRIT | peptide + FC5 sdAb + EA2 |
| | WGGDNTFYSNSVKGRFTISRDNAKNTVYLQMNSLK | sequon (aa 153-165 of rat |
| | PEDTADYYCAAGSTSTATPLRVDYWGKGTQVTVSS | Muc10) + GTG linker + poly- |
| | PTTDSTTPAPTTKGTGHHHHHHGT | His-Tag |
| SEQ ID NO: 25 | MEFGLSWLFLVAILKGVQCDVQLQASGGGLVQAGG | FC5OG1H6: Secretion signal |
| | SLRLSCAASGFKITHYTMGWFRQAPGKEREFVSRIT | peptide + FC5 sdAb + |
| | WGGDNTFYSNSVKGRFTISRDNAKNTVYLQMNSLK | synthetic sequon based on |
| | PEDTADYYCAAGSTSTATPLRVDYWGKGTQVTVSS | Gerken et al. (2006) + GT |
| | FFPTPGPGTHHHHHHGT | linker + poly-His-Tag |
| SEQ ID NO: 26 | MEFGLSWLFLVAILKGVQCDVQLQASGGGLVQAGG | FC5OG2H6: Secretion signal |
| | SLRLSCAASGFKITHYTMGWFRQAPGKEREFVSRIT | peptide + FC5 sdAb + GGG |
| | WGGDNTFYSNSVKGRFTISRDNAKNTVYLQMNSLK | linker + synthetic sequon |
| | PEDTADYYCAAGSTSTATPLRVDYWGKGTQVTVSS | based on Gerken et al. (2006) |
| | GGGFFPTPGPGTHHHHHHGT | + GTG linker + poly-His-Tag |
| SEQ ID NO: 27 | MEFGLSWLFLVAILKGVQCDVQLQASGGGLVQAGG | FC5OG3H6: Secretion signal |
| | SLRLSCAASGFKITHYTMGWFRQAPGKEREFVSRIT | peptide + FC5 sdAb + GGG |
| | WGGDNTFYSNSVKGRFTISRDNAKNTVYLQMNSLK | linker + two synthetic sequons |
| | PEDTADYYCAAGSTSTATPLRVDYWGKGTQVTVSS | based on Gerken et al. (2006) |
| | GGGFFPTPGPFFPTPGPGTHHHHHHGT | + GT linker + poly-His-Tag |
| SEQ ID NO: 28 | MEFGLSWLFLVAILKGVQCDVQLQASGGGLVQAGG | FC5OG4H6: Secretion signal |
| | SLRLSCAASGFKITHYTMGWFRQAPGKEREFVSRIT | peptide + FC5 sdAb + GGG |
| | WGGDNTFYSNSVKGRFTISRDNAKNTVYLQMNSLK | linker + O-linked sequon from |
| | PEDTADYYCAAGSTSTATPLRVDYWGKGTQVTVSS | interferon alpha 2 + GT linker |
| | GGGGVGVTETPLGTHHHHHHGT | + poly-His-Tag |
| SEQ ID NO: 29 | MEFGLSWLFLVAILKGVQCDVQLQASGGGLVQAGG | FC5OG5H6: Secretion signal |
| | SLRLSCAASGFKITHYTMGWFRQAPGKEREFVSRIT | peptide + FC5 sdAb + GGG |
| | WGGDNTFYSNSVKGRFTISRDNAKNTVYLQMNSLK | linker + Shortest sequence |
| | PEDTADYYCAAGSTSTATPLRVDYWGKGTQVTVSS | motif for high level mucin-type |
| | GGGAAATPAPGGGTHHHHHHGT | O-glycosylation based on |
| | | Yoshida et al. (1997) + GGGT |
| | | linker + poly-His-Tag |
| SEQ ID NO: 30 | MEFGLSWVFLVAILKGVQCQVQLVESGGGLAQAGG | A20.1-OG2H6: Secretion |
| | SLRLSCAASGRTFSMDPMAWFRQPPGKEREFVAAG | signal peptide + A20.1 sdAb + |
| | SSTGRTTYYADSVKGRFTISRDNAKNTVYLQMNSLK | GGG linker + synthetic |
| | PEDTAVYYCAAAPYGANWYRDEYAYWGQGTQVTV | sequon based on Gerken et |
| | SSGGGFFPTPGPGTHHHHHHGT | al. + GT linker + poly-His-Tag |
| SEQ ID NO: 31 | MEFGLSWVFLVAILKGVQCQVKLEESGGGLVQAGG | IGF1R5-OG2H6: Secretion |
| | SLRLSCAASGRTIDNYAMAWSRQAPGKDREFVATID | signal peptide + IGF1R5 sdAb |
| | WGDGGARYANSVKGRFTISRDNAKGTMYLQMNNLE | + GGG linker + synthetic |
| | PEDTAVYSCAMARQSRVNLDVARYDYWGQGTQVT | sequon based on Gerken et |
| | VSSGGGFFPTPGPGTHHHHHHGT | al. (2006) + GT linker + poly- |
| | | His-Tag |
| SEQ ID NO: 32 | MEFGLSWVFLVAILKGVQCQVKLEESGGGLVQAGG | IR5hFc1X0OG2: Secretion |
| | SLRLSCAASGRTIDNYAMAWSRQAPGKDREFVATI | signal peptide + IGF1R5 sdAb |
| | DWGDGGARYANSVKGRFTISRDNAKGTMYLQMNN | + hFc1X0 + GGG linker + |
| | LEPEDTAVYSCAMARQSRVNLDVARYDYWGQGTQ | synthetic sequon based on |
| | VTVSSEPKSSDKTHTCPPCPAPELLGGPSVFLFPPK | Gerken et al. (2006) + GT |
| | PKDTLMISRTPEVTCVVVDVSHEGPEVKFNWHVDGV | linker + poly-His-Tag |
| | EVHNAKTKPREEQYNSTYRVVSVLTVLHQDWLNGK | |
| | EYKCKVSNKALPAPIEKTISKAKGQPREPQVYTLPPS | |
| | RDELTKNQVSLTCLVKGFYPSDIAVEWESNGQPENN | |
| | YKTTPPVLDSDGSFFLYSKLTVDKSRWQQGNVFSCS | |
| | VMHEGLHNHYTQKSLSLSPGGGGGFFPTPGP | |
| SEQ ID NO: 33 | MEFGLSWVFLVAILKGVQCQVKLEESGGGLVQAGG | IR5hFc1X0OG6: Secretion |
| | SLRLSCAASGRTIDNYAMAWSRQAPGKDREFVATI | signal peptide + IGF1R5 sdAb |
| | DWGDGGARYANSVKGRFTISRDNAKGTMYLQMNN | + hFc1X0 + GGG linker + O- |
| | LEPEDTAVYSCAMARQSRVNLDVARYDYWGQGTQ | linked sequon present in G- |
| | VTVSSEPKSSDKTHTCPPCPAPELLGGPSVFLFPPK | CSF + GT linker + poly-His- |
| | PKDTLMISRTPEVTCVVVDVSHEGPEVKFNWHVDGV | Tag |
| | EVHNAKTKPREEQYNSTYRVVSVLTVLHQDWLNGK | |
| | EYKCKVSNKALPAPIEKTISKAKGQPREPQVYTLPPS | |
| | RDELTKNQVSLTCLVKGFYPSDIAVEWESNGQPENN | |
| | YKTTPPVLDSDGSFFLYSKLTVDKSRWQQGNVFSCS | |
| | VMHEGLHNHYTQKSLSLSPGGGGGAPALQPTQGA | |
| | MPA | |
| SEQ ID NO: 34 | CATATGGCTATTGGCGAATTCGGATCCTCTAGAGT | pCWori+(−lacZ) |
| | CGACCTGCAGAAGCTTATCGATGATAAGCTGTCAA | |
| | ACATGAGCAGATCTGAGCCCGCCTAATGAGCGGG | |
| | CTTTTTTTTCAGATCTGCTTGAAGACGAAAGGGCC | |
| | TCGTGATACGCCTATTTTTATAGGTTAATGTCATGA | |
| | TAATAATGGTTTCTTAGCGTCAAAGCAACCATAGTA | |
| | CGCGCCCTGTAGCGGCGCATTAAGCGCGGCGGG | |
| | TGTGGTGGTTACGCGCAGCGTGACCGCTACACTT | |
| | GCCAGCGCCCTAGCGCCCGCTCCTTTCGCTTTCT | |
| | TCCCTTCCTTTCTCGCCACGTTCGCCGGCTTTCCC | |
| | CGTCAAGCTCTAAATCGGGGGCTCCCTTTAGGGTT | |
| | CCGATTTAGTGCTTTACGGCACCTCGACCCCAAAA | |
| | AACTTGATTTGGGTGATGGTTCACGTAGTGGGCCA | |
| | TCGCCCTGATAGACGGTTTTTCGCCCTTTGACGTT | |
| | GGAGTCCACGTTCTTTAATAGTGGACTCTTGTTCC | |
| | AAACTGGAACAACACTCAACCCTATCTCGGGCTAT | |
| | TCTTTTGATTTATAAGGGATTTTGCCGATTTCGGCC | |
| | TATTGGTTAAAAAATGAGCTGATTTAACAAAAATTT | |
| | AACGCGAATTTTAACAAAATATTAACGTTTACAATT | |
| | TCAGGTGGCACTTTTCGGGGAAATGTGCGCGGAA | |
| | CCCCTATTTGTTTATTTTTCTAAATACATTCAAATAT | |
| | GTATCCGCTCATGAGACAATAACCCTGATAAATGC | |
| | TTCAATAATATTGAAAAAGGAAGAGTATGAGTATTC | |
| | AACATTTCCGTGTCGCCCTTATTCCCTTTTTTGCG | |
| | GCATTTTGCCTTCCTGTTTTTGCTCACCCAGAAAC | |
| | GCTGGTGAAAGTAAAAGATGCTGAAGATCAGTTGG | |
| | GTGCACGAGTGGGTTACATCGAACTGGATCTCAA | |
| | CAGCGGTAAGATCCTTGAGAGTTTTCGCCCCGAA | |
| | GAACGTTTTCCAATGATGAGCACTTTTAAAGTTCTG | |
| | CTATGTGGCGCGGTATTATCCCGTGTTGACGCCG | |
| | GGCAAGAGCAACTCGGTCGCCGCATACACTATTC | |
| | TCAGAATGACTTGGTTGAGTACTCACCAGTCACAG | |
| | AAAAGCATCTTACGGATGGCATGACAGTAAGAGAA | |
| | TTATGCAGTGCTGCCATAACCATGAGTGATAACAC | |
| | TGCGGCCAACTTACTTCTGACAACGATCGGAGGA | |
| | CCGAAGGAGCTAACCGCTTTTTTGCACAACATGGG | |
| | GGATCATGTAACTCGCCTTGATCGTTGGGAACCG | |
| | GAGCTGAATGAAGCCATACCAAACGACGAGCGTG | |
| | ACACCACGATGCCTGCAGCAATGGCAACAACGTT | |
| | GCGCAAACTATTAACTGGCGAACTACTTACTCTAG | |
| | CTTCCCGGCAACAATTAATAGACTGGATGGAGGC | |
| | GGATAAAGTTGCAGGACCACTTCTGCGCTCGGCC | |
| | CTTCCGGCTGGCTGGTTTATTGCTGATAAATCTGG | |
| | AGCCGGTGAGCGTGGGTCTCGCGGTATCATTGCA | |
| | GCACTGGGGCCAGATGGTAAGCCCTCCCGTATCG | |
| | TAGTTATCTACACGACGGGGAGTCAGGCAACTATG | |
| | GATGAACGAAATAGACAGATCGCTGAGATAGGTG | |
| | CCTCACTGATTAAGCATTGGTAACTGTCAGACCAA | |
| | GTTTACTCATATATACTTTAGATTGATTTAAAACTTC | |
| | ATTTTTAATTTAAAAGGATCTAGGTGAAGATCCTTT | |
| | TTGATAATCTCATGACCAAAATCCCTTAACGTGAGT | |
| | TTTCGTTCCACTGAGCGTCAGACCCCGTAGAAAAG | |
| | ATCAAAGGATCTTCTTGAGATCCTTTTTTTCTGCGC | |
| | GTAATCTGCTGCTTGCAAACAAAAAAACCACCGCT | |
| | ACCAGCGGTGGTTTGTTTGCCGGATCAAGAGCTA | |
| | CCAACTCTTTTTCCGAAGGTAACTGGCTTCAGCAG | |
| | AGCGCAGATACCAAATACTGTCCTTCTAGTGTAGC | |
| | CGTAGTTAGGCCACCACTTCAAGAACTCTGTAGCA | |
| | CCGCCTACATACCTCGCTCTGCTAATCCTGTTACC | |
| | AGTGGCTGCTGCCAGTGGCGATAAGTCGTGTCTT | |
| | ACCGGGTTGGACTCAAGACGATAGTTACCGGATA | |
| | AGGCGCAGCGGTCGGGCTGAACGGGGGGTTCGT | |
| | GCACACAGCCCAGCTTGGAGCGAACGACCTACAC | |
| | CGAACTGAGATACCTACAGCGTGAGCTATGAGAAA | |
| | GCGCCACGCTTCCCGAAGGGAGAAAGGCGGACA | |
| | GGTATCCGGTAAGCGGCAGGGTCGGAACAGGAG | |
| | AGCGCACGAGGGAGCTTCCAGGGGGAAACGCCT | |
| | GGTATCTTTATAGTCCTGTCGGGTTTCGCCACCTC | |
| | TGACTTGAGCGTCGATTTTTGTGATGCTCGTCAGG | |
| | GGGGCGGAGCCTATGGAAAAACGCCAGCAACGC | |
| | GGCCTTTTTACGGTTCCTGGCCTTTTGCTGGCCTT | |
| | TTGCTCACATGTTCTTTCCTGCGTTATCCCCTGATT | |
| | CTGTGGATAACCGTATTACCGCCTTTGAGTGAGCT | |
| | GATACCGCTCGCCGCAGCCGAACGACCGAGCGC | |
| | AGCGAGTCAGTGAGCGAGGAAGCGGAAGAGCGC | |
| | CTGATGCGGTATTTTCTCCTTACGCATCTGTGCGG | |
| | TATTTCACACCGCATATATGGTGCACTCTCAGTAC | |
| | AATCTGCTCTGATGCCGCATAGTTAAGCCAGTATA | |
| | CACTCCGCTATCGCTACGTGACTGGGTCATGGCT | |
| | GCGCCCCGACACCCGCCAACACCCGCTGACGCG | |
| | CCCTGACGGGCTTGTCTGCTCCCGGCATCCGCTT | |
| | ACAGACAAGCTGTGACCGTCTCCGGGAGCTGCAT | |
| | GTGTCAGAGGTTTTCACCGTCATCACCGAAACGCC | |
| | ATACGAGCCGGAAGCATAAAGTGTAAAGCCTGGG | |
| | GTGCCTAATGAGTGAGCTAACTCACATTAATTGCG | |
| | TTGCGCTCACTGCCCGCTTTCCAGTCGGGAAACC | |
| | TGTCGTGCCAGCTGCATTAATGAATCGGCCAACG | |
| | CGCGGGGAGAGGCGGTTTGCGTATTGGGCGCCA | |
| | GGGTGGTTTTTCTTTTCACCAGTGAGACGGGCAAC | |
| | AGCTGATTGCCCTTCACCGCCTGGCCCTGAGAGA | |
| | GTTGCAGCAAGCGGTCCACGCTGGTTTGCCCCAG | |
| | CAGGCGAAAATCCTGTTTGATGGTGGTTAACGGC | |
| | GGGATATAACATGAGCTGTCTTCGGTATCGTCGTA | |
| | TCCCACTACCGAGATATCCGCACCAACGCGCAGC | |
| | CCGGACTCGGTAATGGCGCGCATTGCGCCCAGCG | |
| | CCATCTGATCGTTGGCAACCAGCATCGCAGTGGG | |
| | AACGATGCCCTCATTCAGCATTTGCATGGTTTGTT | |
| | GAAAACCGGACATGGCACTCCAGTCGCCTTCCCG | |
| | TTCCGCTATCGGCTGAATTTGATTGCGAGTGAGAT | |
| | ATTTATGCCAGCCAGCCAGACGCAGACGCGCCGA | |
| | GACAGAACTTAATGGGCCCGCTAACAGCGCGATT | |
| | TGCTGGTGACCCAATGCGACCAGATGCTCCACGC | |
| | CCAGTCGCGTACCGTCTTCATGGGAGAAAATAATA | |
| | CTGTTGATGGGTGTCTGGTCAGAGACATCAAGAAA | |
| | TAACGCCGGAACATTAGTGCAGGCAGCTTCCACA | |
| | GCAATGGCATCCTGGTCATCCAGCGGATAGTTAAT | |
| | GATCAGCCCACTGACGCGTTGCGCGAGAAGATTG | |
| | TGCACCGCCGCTTTACAGGCTTCGACGCCGCTTC | |
| | GTTCTACCATCGACACCACCACGCTGGCACCCAG | |
| | TTGATCGGCGCGAGATTTAATCGCCGCGACAATTT | |
| | GCGACGGCGCGTGCAGGGCCAGACTGGAGGTGG | |
| | CAACGCCAATCAGCAACGACTGTTTGCCCGCCAG | |
| | TTGTTGTGCCACGCGGTTGGGAATGTAATTCAGCT | |
| | CCGCCATCGCCGCTTCCACTTTTTCCCGCGTTTTC | |
| | GCAGAAACGTGGCTGGCCTGGTTCACCACGCGGG | |
| | AAACGGTCTGATAAGAGACACCGGCATACTCTGC | |
| | GACATCGTATAACGTTACTGGTTTCACATTCACCA | |
| | CCCTGAATTGACTCTCTTCCGGGCGCTATCATGCC | |
| | ATACCGCGAAAGGTTTTGCGCCATTCGATGGTGTC | |
| | CTGGCACGACAGGTTTCCCGACTGGAAAGCGGGC | |
| | AGTGAGCGCAACGCAATTAATGTGAGTTAGCTCAC | |
| | TCATTAGGCACCCCAGGCTTTACACTTTATGCTTC | |
| | CGGCTCGTATAATGTGTGGAATTGTGAGCGGATAA | |
| | CAATTTCACACAGGAAACAGGATCGATCCATCGAT | |
| | GAGCTTACTCCCCATCCCCCTGTTGACAATTAATC | |
| | ATCGGCTCGTATAATGTGTGGAATTGTGAGCGGAT | |
| | AACAATTTCACACAGGAAACAGGATCAGCTTACTC | |
| | CCCATCCCCCTGTTGACAATTAATCATCGGCTCGT | |
| | ATAATGTGTGGAATTGTGAGCGGATAACAATTTCA | |
| | CACAGGAAACAGGATCCATCGATGCTTAGGAGGT | |
| SEQ ID NO: 35 | GTACATTTATATTGGCTCATGTCCAATATGACCGC | pTT5 |
| | CATGTTGACATTGATTATTGACTAGTTATTAATAGT | |
| | AATCAATTACGGGGTCATTAGTTCATAGCCCATAT | |
| | ATGGAGTTCCGCGTTACATAACTTACGGTAAATGG | |
| | CCCGCCTGGCTGACCGCCCAACGACCCCCGCCC | |
| | ATTGACGTCAATAATGACGTATGTTCCCATAGTAA | |
| | CGCCAATAGGGACTTTCCATTGACGTCAATGGGTG | |
| | GAGTATTTACGGTAAACTGCCCACTTGGCAGTACA | |
| | TCAAGTGTATCATATGCCAAGTCCGCCCCCTATTG | |
| | ACGTCAATGACGGTAAATGGCCCGCCTGGCATTAT | |
| | GCCCAGTACATGACCTTACGGGACTTTCCTACTTG | |
| | GCAGTACATCTACGTATTAGTCATCGCTATTACCAT | |
| | GGTGATGCGGTTTTGGCAGTACACCAATGGGCGT | |
| | GGATAGCGGTTTGACTCACGGGGATTTCCAAGTCT | |
| | CCACCCCATTGACGTCAATGGGAGTTTGTTTTGGC | |
| | ACCAAAATCAACGGGACTTTCCAAAATGTCGTAAT | |
| | AACCCCGCCCCGTTGACGCAAATGGGGGGTAGGC | |
| | GTGTACGGTGGGAGGTCTATATAAGCAGAGCTCG | |
| | TTTAGTGAACCGTCAGATCCTCACTCTCTTCCGCA | |
| | TCGCTGTCTGCGAGGGCCAGCTGTTGGGCTCGCG | |
| | GTTGAGGACAAACTCTTCGCGGTCTTTCCAGTACT | |
| | CTTGGATCGGAAACCCGTCGGCCTCCGAACGGTA | |
| | CTCCGCCACCGAGGGACCTGAGCGAGTCCGCATC | |
| | GACCGGATCGGAAAACCTCTCGAGAAAGGCGTCT | |
| | AACCAGTCACAGTCGCAAGGTAGGCTGAGCACCG | |
| | TGGCGGGCGGCAGCGGGTGGCGGTCGGGGTTGT | |
| | TTCTGGCGGAGGTGCTGCTGATGATGTAATTAAAG | |
| | TAGGCGGTCTTGAGACGGCGGATGGTCGAGGTGA | |
| | GGTGTGGCAGGCTTGAGATCCAGCTGTTGGGGTG | |
| | AGTACTCCCTCTCAAAAGCGGGCATTACTTCTGCG | |
| | CTAAGATTGTCAGTTTCCAAAAACGAGGAGGATTT | |
| | GATATTCACCTGGCCCGATCTGGCCATACACTTGA | |
| | GTGACAATGACATCCACTTTGCCTTTCTCTCCACA | |
| | GGTGTCCACTCCCAGGTCCAAGTTTAAACGGATCT | |
| | CTAGCGAATTCCCTCTAGAGGGCCCGTTTCTGCTA | |
| | GCAAGCTTGCTAGCGGCCGCTCGAGGCCGGCAA | |
| | GGCCGGATCCCCCGACCTCGACCTCTGGCTAATA | |
| | AAGGAAATTTATTTTCATTGCAATAGTGTGTTGGAA | |
| | TTTTTTGTGTCTCTCACTCGGAAGGACATATGGGA | |
| | GGGCAAATCATTTGGTCGAGATCCCTCGGAGATCT | |
| | CTAGCTAGAGGATCGATCCCCGCCCCGGACGAAC | |
| | TAAACCTGACTACGACATCTCTGCCCCTTCTTCGC | |
| | GGGGCAGTGCATGTAATCCCTTCAGTTGGTTGGTA | |
| | CAACTTGCCAACTGAACCCTAAACGGGTAGCATAT | |
| | GCTTCCCGGGTAGTAGTATATACTATCCAGACTAA | |
| | CCCTAATTCAATAGCATATGTTACCCAACGGGAAG | |
| | CATATGCTATCGAATTAGGGTTAGTAAAAGGGTCC | |
| | TAAGGAACAGCGATGTAGGTGGGCGGGCCAAGAT | |
| | AGGGGCGCGATTGCTGCGATCTGGAGGACAAATT | |
| | ACACACACTTGCGCCTGAGCGCCAAGCACAGGGT | |
| | TGTTGGTCCTCATATTCACGAGGTCGCTGAGAGCA | |
| | CGGTGGGCTAATGTTGCCATGGGTAGCATATACTA | |
| | CCCAAATATCTGGATAGCATATGCTATCCTAATCTA | |
| | TATCTGGGTAGCATAGGCTATCCTAATCTATATCT | |
| | GGGTAGCATATGCTATCCTAATCTATATCTGGGTA | |
| | GTATATGCTATCCTAATTTATATCTGGGTAGCATAG | |
| | GCTATCCTAATCTATATCTGGGTAGCATATGCTATC | |
| | CTAATCTATATCTGGGTAGTATATGCTATCCTAATC | |
| | TGTATCCGGGTAGCATATGCTATCCTAATAGAGAT | |
| | TAGGGTAGTATATGCTATCCTAATTTATATCTGGGT | |
| | AGCATATACTACCCAAATATCTGGATAGCATATGC | |
| | TATCCTAATCTATATCTGGGTAGCATATGCTATCCT | |
| | AATCTATATCTGGGTAGCATAGGCTATCCTAATCTA | |
| | TATCTGGGTAGCATATGCTATCCTAATCTATATCTG | |
| | GGTAGTATATGCTATCCTAATTTATATCTGGGTAGC | |
| | ATAGGCTATCCTAATCTATATCTGGGTAGCATATG | |
| | CTATCCTAATCTATATCTGGGTAGTATATGCTATCC | |
| | TAATCTGTATCCGGGTAGCATATGCTATCCTCATG | |
| | ATAAGCTGTCAAACATGAGAATTAATTCTTGAAGAC | |
| | GAAAGGGCCTCGTGATACGCCTATTTTTATAGGTT | |
| | AATGTCATGATAATAATGGTTTCTTAGACGTCAGGT | |
| | GGCACTTTTCGGGGAAATGTGCGCGGAACCCCTA | |
| | TTTGTTTATTTTTCTAAATACATTCAAATATGTATCC | |
| | GCTCATGAGACAATAACCCTGATAAATGCTTCAAT | |
| | AATATTGAAAAAGGAAGAGTATGAGTATTCAACATT | |
| | TCCGTGTCGCCCTTATTCCCTTTTTTGCGGCATTTT | |
| | GCCTTCCTGTTTTTGCTCACCCAGAAACGCTGGTG | |
| | AAAGTAAAAGATGCTGAAGATCAGTTGGGTGCACG | |
| | AGTGGGTTACATCGAACTGGATCTCAACAGCGGTA | |
| | AGATCCTTGAGAGTTTTCGCCCCGAAGAACGTTTT | |
| | CCAATGATGAGCACTTTTAAAGTTCTGCTATGTGG | |
| | CGCGGTATTATCCCGTGTTGACGCCGGGCAAGAG | |
| | CAACTCGGTCGCCGCATACACTATTCTCAGAATGA | |
| | CTTGGTTGAGTACTCACCAGTCACAGAAAAGCATC | |
| | TTACGGATGGCATGACAGTAAGAGAATTATGCAGT | |
| | GCTGCCATAACCATGAGTGATAACACTGCGGCCA | |
| | ACTTACTTCTGACAACGATCGGAGGACCGAAGGA | |
| | GCTAACCGCTTTTTTGCACAACATGGGGGATCATG | |
| | TAACTCGCCTTGATCGTTGGGAACCGGAGCTGAAT | |
| | GAAGCCATACCAAACGACGAGCGTGACACCACGA | |
| | TGCCTGCAGCAATGGCAACAACGTTGCGCAAACT | |
| | ATTAACTGGCGAACTACTTACTCTAGCTTCCCGGC | |
| | AACAATTAATAGACTGGATGGAGGCGGATAAAGTT | |
| | GCAGGACCACTTCTGCGCTCGGCCCTTCCGGCTG | |
| | GCTGGTTTATTGCTGATAAATCTGGAGCCGGTGAG | |
| | CGTGGGTCTCGCGGTATCATTGCAGCACTGGGGC | |
| | CAGATGGTAAGCCCTCCCGTATCGTAGTTATCTAC | |
| | ACGACGGGGAGTCAGGCAACTATGGATGAACGAA | |
| | ATAGACAGATCGCTGAGATAGGTGCCTCACTGATT | |
| | AAGCATTGGTAACTGTCAGACCAAGTTTACTCATA | |
| | TATACTTTAGATTGATTTAAAACTTCATTTTTAATTT | |
| | AAAAGGATCTAGGTGAAGATCCTTTTTGATAATCTC | |
| | ATGACCAAAATCCCTTAACGTGAGTTTTCGTTCCA | |
| | CTGAGCGTCAGACCCCGTAGAAAAGATCAAAGGA | |
| | TCTTCTTGAGATCCTTTTTTTCTGCGCGTAATCTGC | |
| | TGCTTGCAAACAAAAAAACCACCGCTACCAGCGGT | |
| | GGTTTGTTTGCCGGATCAAGAGCTACCAACTCTTT | |
| | TTCCGAAGGTAACTGGCTTCAGCAGAGCGCAGAT | |
| | ACCAAATACTGTTCTTCTAGTGTAGCCGTAGTTAG | |
| | GCCACCACTTCAAGAACTCTGTAGCACCGCCTACA | |
| | TACCTCGCTCTGCTAATCCTGTTACCAGTGGCTGC | |
| | TGCCAGTGGCGATAAGTCGTGTCTTACCGGGTTG | |
| | GACTCAAGACGATAGTTACCGGATAAGGCGCAGC | |
| | GGTCGGGCTGAACGGGGGGTTCGTGCACACAGC | |
| | CCAGCTTGGAGCGAACGACCTACACCGAACTGAG | |
| | ATACCTACAGCGTGAGCTATGAGAAAGCGCCACG | |
| | CTTCCCGAAGGGAGAAAGGCGGACAGGTATCCGG | |
| | TAAGCGGCAGGGTCGGAACAGGAGAGCGCACGA | |
| | GGGAGCTTCCAGGGGGAAACGCCTGGTATCTTTA | |
| | TAGTCCTGTCGGGTTTCGCCACCTCTGACTTGAGC | |
| | GTCGATTTTTGTGATGCTCGTCAGGGGGGCGGAG | |
| | CCTATGGAAAAACGCCAGCAACGCGGCCTTTTTAC | |
| | GGTTCCTGGCCTTTTGCTGGCCTTTTGCTCACATG | |
| | TTCTTTCCTGCGTTATCCCCTGATTCTGTGGATAAC | |
| | CGTATTACCGCCTTTGAGTGAGCTGATACCGCTCG | |
| | CCGCAGCCGAACGACCGAGCGCAGCGAGTCAGT | |
| | GAGCGAGGAAGC | |
| SEQ ID NO: 36 | DIVMSQSPSSLAVSAGERVTMSCKSSQSLLNSRTRK | Light chain of Anti-CD3 Fab |
| | NYLAWYQQKPGQSPKLLIYWASTRESGVPDRFTGS | |
| | GSGTDFTLTISSVQAEDLAVYYCIQSYHLRTFGGGTK | |
| | LEIKRTVAAPSVFIFPPSDEQLKSGTASVVCLLNNFYP | |
| | REAKVQWKVDNALQSGNSQESVTEQDSKDSTYSLS | |
| | STLTLSKADYEKHKVYACEVTHQGLSSPVTKSFNRG | |
| | EC | |
| SEQ ID NO: 37 | QVQLQQPGAELGKPGTSVKLSCKASGYTFTSYWIH | Heavy chain of Anti-CD3 Fab |
| | WVKQRPGQGLEWVGNINPNSGSINYNEKFKNKATL | |
| | TVDKSSSTAYMQLSTLTSEDFAVYYCTRDTSGQYYF | |
| | DYWGQGTTLTVSSASTKGPSVFPLAPSSKSTSGGTA | |
| | ALGCLVKDYFPEPVTVSWNSGALTSGVHTFPAVLQS | |
| | SGLYSLSSVVTVPSSSLGTQTYICNVNHKPSNTKVDK | |
| | KVEPKSCDGGGGGAPALQPTQGAMPA | |
| SEQ ID NO: 38 | GWTLNSAGYLLGPHAIDNHRSFSDKHGLT | Rat Galanin |
| SEQ ID NO: 39 | GWTLNSAGYLLGPHAIDNHRSFSDKHGLT-Cya | Rat galanin with a cysteamide |
| | | modification (Cya) at the C- |
| | | terminus |

Legend: underlined = sequon region; italic-underlined = Secretion signal peptide; Bold: repeated sequon; Bold-underlined = IGF1R5 sdAb.

### REFERENCES

Abulrob A, Zhang J, Tanha J, MacKenzie R & Stanimirovic D (2005) International Congress Series 1277: 212-223.
Arbabi-Ghahroudi, M., Desmyter, A., Wyns, L., Hamers, R., Muyldermans, S. (1997) FEBS Lett 414:521-6.
Bell, A., Wang, Z. J., Arbabi-Ghahroudi, M., Chang, T. A., Durocher, Y., Trojahn, U., Baardsnes, J., Jaramillo, M. L., Li, S., Baral, T. N., O'Connor-McCourt, M., MacKenzie, R., Zhang, J. (2010) Cancer Lett 289:81-90.
Chothia, C., Lesk, A. M. (1987) J Mol Biol 196:901-17.
Davies, J., Riechmann, L. (1996) Immunotechnology 2:169-79.
de Kruif, J., Logtenberg, T. (1996) J Biol Chem 271:7630-4.
Dumoulin, M., Conrath, K., Van Meirhaeghe, A., Meersman, F., Heremans, K., Frenken, L. G., Muyldermans, S., Wyns, L., Matagne, A. (2002) Protein Sci 11:500-15.
Eisenberg, D., Schwarz, E., Komaromy, M., and Wall, R. (1984). J. Mol. Biol. 179:125-42.
Gerken T A, Raman J, Fritz T A & Jamison O (2006) J Biol Chem 281: 32403-32416.
Hamers-Casterman, C., Atarhouch, T., Muyldermans, S., Robinson, G., Hamers, C., Songa, E. B., Bendahman, N., Hamers, R. (1993) Nature 363:446-8.
Hussack, G., Arbabi-Ghahroudi, M., van Faassen, H., Songer, J. G., Ng, K. K., MacKenzie, R., Tanha, J. (2011a) J Biol Chem 286:8961-76.
Hussack, G., Hirama, T., Ding, W., MacKenzie, R., Tanha, J. (2011 b) PLoS One 6:e28218.
Iqbal, U, Trojahn, U., Albaghdadi, H., Zhang, J., O'Connor-McCourt, M., Stanimirovic D., Tomanek, B., Sutherland, G., Abulrob, A. (2010) Br J Pharmacol 160:1016-28.
Jespers, L., Schon, O., James, L. C., Veprintsev, D., Winter G. (2004) J Mol Biol 337:893-903.
Kabat, E. A., Wu, T. T., (1991) J Immunol 147:1709-1719.
Kim, D. Y., Kandalaft, H., Ding, W., Ryan, S., van Faassen, H., Hirama, T., Foote, S. J., MacKenzie, R., Tanha, J. (2012) Protein Eng Des Sel 25:581-9.
Li, S., Zheng, W., Kuolee, R., Hirama, T., Henry, M., Makvandi-Nejad, S., Fjsllman, T., Chen, W., Zhang, J. (2009) Mol Immunol 46:1718-26.
Merritt, E. A., Hol, W. G. (1995) Curr Opin Struct Biol 5:165-71.
Nicaise, M., Valerio-Lepiniec, M., Minard, P., Desmadril, M. (2004) Protein Sci 13:1882-91.
Nielsen, U. B., Adams, G. P., Weiner, L. M., Marks, J. D. (2000) Cancer Res 60:6434-40.
Nuttall, S. D., Krishnan, U. V., Doughty, L., Pearson, K., Ryan, M. T., Hoogenraad, N.J., Hattarki, M., Carmichael, J. A., Irving, R. A., Hudson, P. J. (2003) Eur J Biochem 270:3543-54.
Ridgway, J. B., Presta, L. G., Carter, P. (1996) Protein Eng 9:617-21.
Spiess, C, Zhai, Q., Carter, P. J. (2015) Mol Immunol 67:95-106.
To, R., Hirama, T., Arbabi-Ghahroudi, M., MacKenzie, R., Wang, P., Xu, P., Ni, F., Tanha, J. (2005) J Biol Chem 280:41395-403.
Yoshida A, Suzuki M, Ikenaga H & Takeuchi M (1997) J Biol Chem 272: 16884-16888.
Zhang, J., Li, Q., Nguyen, T. D., Tremblay, T. L., Stone, E., To, R., Kelly, J., MacKenzie C. R. (2004a) J Mol Biol 341:161-9.
Zhang, J., Tanha, J., Hirama, T., Khieu, N. H., To, R., Tong-Sevinc, H., Stone, E., Brisson, J. R., MacKenzie, C. R. (2004b) J Mol Biol 335:49-56.
Zhu, X., Wang, L., Liu, R., Flutter, B., Li, S., Ding, J., Tao, H., Liu, C., Sun, M., Gao, B. (2010) Immunol Cell Biol 88:667-75.

1. An antibody or antigen-binding fragment that binds specifically to a target antigen, wherein the antibody or antigen-binding fragment comprises complementarity determining regions (CDR1, CDR2 and CDR3) and an added O-glycosylation sequon glycosylated with an O-glycan having the general formula (I): 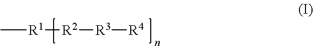
wherein
R¹ is an initial N-acetylgalactosamine (GalNAc);
n=1, or 2;
R² are each independently absent, galactose (Gal), GalNAc, N-Acetylglucosamine (GlcNAc), or a sialic acid;
R³ are each independently absent, Gal or a sialic acid; and
R⁴ are each independently absent or a sialic acid.

2. The antibody or antigen-binding fragment of claim 1, wherein said initial GalNAc, and/or any one of said R¹, R², R³ and R⁴ is further modified with one or more compound.

3. The antibody or antigen-binding fragment of claim 2, wherein said compound is one or more of a methyl group, an acetyl group, a sulfate group, or a combination thereof.

4. The antibody or antigen-binding fragment of claim 1, wherein said sialic acid is N-Acetylneuraminic acid (Neu5Ac), 9-azido-N-Acetylneuraminic acid (9N₃-Neu5Ac), N-azidoacetylneuraminic acid (Neu5NAz), or a combination thereof.

5. The antibody or antigen-binding fragment of any one of claims 1-4, wherein n=1 and R² is Gal.

6. The antibody or antigen-binding fragment of claim 5, wherein R³ is a sialic acid selected from the group consisting of Neu5Ac, Neu5NAz and 9N₃-Neu5Ac, and R⁴ is absent.

7. The antibody or antigen-binding fragment of claim 1, wherein the O-glycan has the general formula (II): 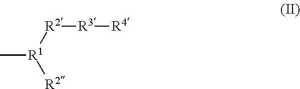
wherein
R^{2′} is Gal, or GlcNAc;
R^{3′} is Gal or a sialic acid;
R^{4′} is absent, or a sialic acid; and
R^{2″} is GlcNAc or a sialic acid.

8. The antibody or antigen-binding fragment of claim 7, wherein
a R^{2′} is Gal, a R^{3′} is a sialic acid consisting of Neu5Ac, and a R^{4′} is absent or a sialic acid consisting of 9N₃-Neu5Ac; and
a R^{2″} is a sialic acid consisting of Neu5Ac.

9. The antibody or antigen-binding fragment of any one of claims 1 to 8, wherein said added O-glycosylation sequon comprise an amino acid sequence comprising:
(SEQ ID NO: 1)
PTTDSTX₁PAPTTK, where X₁ is S or T;
(SEQ ID NO: 2)
FFPX₂PGP, where X₂ is S or T;
(SEQ ID NO: 3)
GVGVX₃ETP, where X₃ is S or T;
(SEQ ID NO: 4)
AAAX₄PAP, where X₄ is S or T;
and
(SEQ ID NO: 5)
APALQPX₅QGAMPA, where X₅ is S or T,
or
combinations thereof.

10. The antibody or antigen-binding fragment of claim 9, wherein said added O-glycosylation sequon comprise an amino acid sequence comprising:
(SEQ ID NO: 6)
PTTDSTTPAPTTK,
(SEQ ID NO: 7)
PTTDSTSPAPTTK,
(SEQ ID NO: 8)
FFPTPGP;
(SEQ ID NO: 9)
FFPSPGP,
(SEQ ID NO: 10)
GVGVTETP,
(SEQ ID NO: 11)
GVGVSETP,
(SEQ ID NO: 12)
AAATPAP,
(SEQ ID NO: 13)
AAASPAP;
(SEQ ID NO: 14)
APALQPTQGAMPA,
and
(SEQ ID NO: 15)
APALQPSQGAMPA.

11. The antibody or antigen-binding fragment of any one of claims 1 to 10, wherein said added O-glycosylation sequon is at a C-terminus of said antibody or antigen-binding fragment.

12. The antibody or antigen-binding fragment of any one of claims 1 to 11, wherein the antigen-binding fragment is a single-domain antibody (sdAb), a fragment antigen binding (Fab), a single-chain variable fragment (scFv), or a single-chain fragment antigen binding (scFab).

13. The antibody or antigen-binding fragment of any one of claims 1 to 12, wherein the antibody is an IgA, an IgD, an IgE, an IgG, or an IgM.

14. The antibody or antigen-binding fragment of any one of claims 1 to 13, wherein the antibody or antigen-binding fragment is humanized or partially humanized.

15. The antibody or antigen-binding fragment of any one of claims 1 to 14, wherein said added O-glycosylation sequon is linked to a first functional moiety via a peptide linker.

16. The antibody or antigen-binding fragment of any one of claims 1 to 14, wherein the antibody or antigen-binding fragment is functionally linked to a first functional moiety via a peptide linker.

17. The antibody or antigen-binding fragment of claim 15 or 16, wherein the peptide linker comprises about 1 to about 40 amino acid residues.

18. The antibody or antigen-binding fragment of any one of claims 15 to 17, wherein the peptide linker comprises the amino acid sequence (SS)ₙ, (GGG)ₙ, (GGGG)ₙ, (GGGS)ₙ, or (SSGGG)ₙ, wherein n≥1.

19. A compound comprising the antibody or antigen-binding fragment of any one of claims 1 to 18, further comprising a second functional moiety operably linked to said O-glycan.

20. The compound of claim 19, wherein said second functional moiety operably linked to said O-glycan is a peptide, a polypeptide, a protein, an enzyme, a second antibody, an antibody fragment, a second antigen-binding fragment or a combination of any two or more thereof; wherein each of the antibody or antigen-binding fragment thereof and the linked peptide, polypeptide, protein, enzyme, second antibody, antibody fragment, second antigen-binding fragment, or the combination of any two or more thereof is functional.

21. A composition comprising the antibody or antigen-binding fragment of any one of claims 1 to 18 or the compound of claim 19 or 20, and a pharmaceutically acceptable diluent, carrier or excipient.

22. A nucleic acid molecule encoding an antibody or antigen-binding fragment thereof of any one of claims 1 to 18.

23. A vector comprising the nucleic acid molecule of claim 22 operably linked to one or more regulatory elements to allow expression of the antibody or antigen-binding fragment of any one of claims 1 to 18 in a host cell.

24. A cell comprising the vector of claim 23 for expressing the antibody or antigen-binding fragment thereof of any one of claims 1 to 18.

25. A method of glycosylating an antibody or antigen-binding fragment that binds specifically to a target antigen and having an added O-glycosylation sequon, wherein the antibody or antigen-binding fragment comprises complementarity determining regions (CDR1, CDR2 and CDR3), the method comprising step a):
a) reacting said antibody or antigen-binding fragment that binds specifically to a target antigen and having an added O-glycosylation sequon, with polypeptide N-acetylgalactosaminyltransferase 2 (ppGalNAcT2) and UDP-N-acetyl-α-D-galactosamine, to obtain an antibody or antigen-binding fragment that binds specifically to a target antigen having an added O-glycosylation sequon glycosylated with an O-glycan comprising an initial N-acetylgalactosamine (GalNAc).

26. The method of claim 25, further comprising step b):
b) reacting said antibody or antigen-binding fragment that binds specifically to a target antigen having an added O-glycosylation sequon glycosylated with an O-glycan comprising an initial GalNAc, with a β-1,3-galactosyltransferase (β3GalT) enzyme and UDP-galactose, to obtain an antibody or antigen-binding fragment that binds specifically to a target antigen having an added O-glycosylation sequon glycosylated with an O-glycan comprising GalNAc-Gal.

27. The method of claim 26, further comprising step c):
c) reacting said antibody or antigen-binding fragment that binds specifically to a target antigen having an added O-glycosylation sequon glycosylated with an O-glycan comprising GalNAc-Gal, with a β-Galactoside α-2,3-Sialyltransferase 1 enzyme (ST3Gal1) and CMP-9N₃-Neu5Ac, to obtain an antibody or antigen-binding fragment that binds specifically to a target antigen having an added O-glycosylation sequon glycosylated with an O-glycan comprising GalNAc-Gal-9N₃-Neu5Ac.

28. A method of glycosylating an antibody or antigen-binding fragment that binds specifically to a target antigen and having an added O-glycosylation sequon glycosylated with an O-glycan comprising a first sugar comprising GalNAc-Gal-Neu5Ac and a second sugar comprising NeuAc, wherein the antibody or antigen-binding fragment comprises complementarity determining regions (CDR1, CDR2 and CDR3), the method comprising step a):
a) reacting said antibody or antigen-binding fragment that binds specifically to a target antigen and having an added O-glycosylation sequon glycosylated with an O-glycan comprising a first sugar comprising GalNAc-Gal-Neu5Ac and a second sugar comprising NeuAc, with an α-N-Acetyl-Neuraminide α-2,8-Sialyltransferase enzyme and CMP-9N₃-Neu5Ac, to obtain an antibody or antigen-binding fragment that binds specifically to a target antigen having an added O-glycosylation sequon glycosylated with an O-glycan comprising a first sugar comprising GalNAc-Gal-Neu5Ac-9N₃-Neu5Ac and a second sugar comprising NeuAc.

29. A method of glycosylating an antibody or antigen-binding fragment that binds specifically to a target antigen and having an added O-glycosylation sequon glycosylated with an O-glycan comprising a first sugar comprising GalNAc-Gal-Neu5Ac and a second sugar comprising NeuAc, wherein the antibody or antigen-binding fragment comprises complementarity determining regions (CDR1, CDR2 and CDR3), the method comprising step a):
a) reacting said antibody or antigen-binding fragment that binds specifically to a target antigen and having an added O-glycosylation sequon glycosylated with an O-glycan comprising a first sugar comprising GalNAc-Gal-Neu5Ac and a second sugar comprising NeuAc, with a neuraminidase enzyme, to obtain an antibody or antigen-binding fragment that binds specifically to a target antigen having an added O-glycosylation sequon glycosylated with an O-glycan comprising GalNAc-Gal.

30. The method of claim 29, further comprising step b):
b) reacting said antibody or antigen-binding fragment that binds specifically to a target antigen having an added O-glycosylation sequon glycosylated with an O-glycan comprising GalNAc-Gal, with a β-Galactoside α-2,3-Sialyltransferase 1 enzyme (ST3Gal1) and CMP-9N₃-Neu5Ac, to obtain an antibody or antigen-binding fragment that binds specifically to a target antigen having an added O-glycosylation sequon glycosylated with an O-glycan comprising GalNAc-Gal-9N₃-Neu5Ac.

31. A method of producing a compound comprising an antibody or antigen-binding fragment according to any one of claims 4 to 18 and a functional moiety operably linked to said O-glycan, the method comprising the step of conjugating said antibody or antigen-binding fragment comprising an O-glycan comprising a 9N₃-Neu5Ac moiety with a functional moiety comprising a functionalized cyclooctyne, to obtain a compound comprising an antibody or antigen-binding fragment conjugated to said functional moiety operably linked to said O-glycan.

32. The method of claim 31, wherein said functionalized cyclooctyne is cyclooctyne (COT), monofluorinated cyclooctyne (MOFO), difluorocyclooctyne (DIFO), dimethoxyazacyclooctyne (DIMAC), dibenzocyclooctyne (DIBO), dibenzoazacyclooctyne (DIBAC), biarylazacyclooctynone (BARAC), bicyclononyne (BCN), 2,3,6,7-tetramethoxy-DIBO (TMDIBO), sulfonylated DIBO (S-DIBO), carboxymethylmonobenzocyclooctyne (COMBO), pyrrolocyclooctyne (PYRROC), or combinations thereof.

33. The method of any one of claims 25 to 32, wherein said added O-glycosylation sequon comprises an amino acid sequence comprising:
(SEQ ID NO: 1)
PTTDSTX₁PAPTTK, where X₁ is S or T;
(SEQ ID NO: 2)
FFPX₂PGP, where X₂ is S or T;
(SEQ ID NO: 3)
GVGVX₃ETP, where X₃ is S or T;
(SEQ ID NO: 4)
AAAX₄PAP, where X₄ is S or T;
and
(SEQ ID NO: 5)
APALQPX₅QGAMPA, where X₅ is S or T,
or
combinations thereof.

34. The method of claim 33, wherein said added O-glycosylation sequon comprise an amino acid sequence comprising:
(SEQ ID NO: 6)
PTTDSTTPAPTTK,
(SEQ ID NO: 7)
PTTDSTSPAPTTK,
(SEQ ID NO: 8)
FFPTPGP;
(SEQ ID NO: 9)
FFPSPGP,
(SEQ ID NO: 10)
GVGVTETP,
(SEQ ID NO: 11)
GVGVSETP,
(SEQ ID NO: 12)
AAATPAP,
(SEQ ID NO: 13)
AAASPAP;
(SEQ ID NO: 14)
APALQPTQGAMPA,
(SEQ ID NO: 15)
APALQPSQGAMPA,
or
a combination thereof.
